(19) United States
(12) Patent Application Publication
(10) Pub. No.: US 2022/0218835 A1
(43) Pub. Date: Jul. 14, 2022

(54) COMBINATION THERAPY

(71) Applicants: MedImmune, LLC, Gaithersburg, MD (US); Dana-Farber Cancer Institute, Inc., Boston, MA (US)

(72) Inventors: Krista KINNEER, Gaithersburg, MD (US); David Alan TICE, Gaithersburg, MD (US); Steven COATS, Gaithersburg, MD (US); Yu-Tzu TAI, Newton, MA (US); Kenneth ANDERSON, Wellesley, MA (US)

(21) Appl. No.: 17/595,671

(22) PCT Filed: May 29, 2020
(86) PCT No.: PCT/IB2020/055128

§ 371 Date: Nov. 22, 2021

Related U.S. Application Data
(60) Provisional application No. 62/855,040, filed on May 31, 2019.
(60) Provisional application No. 62/944,698, filed on Dec. 6, 2019.

(51) Int. Cl. A61K 47/68; A61K 31/407; A61K 31/427; A61K 31/69; C07K 16/28

(52) U.S. Cl. CPC A61K 47/6803; A61K 31/407; A61K 31/427; A61K 31/69; C07K 2317/92; A61K 47/6889; C07K 16/2878; C07K 2317/73; A61K 47/6849

(57) ABSTRACT

The disclosure relates to methods and compositions for the treatment of a B-cell malignancy. Specifically, the disclosure relates to a B-cell malignancy medicament or composition, comprising: (a) an antibody-drug conjugate (ADC) comprising an antibody or antigen-binding fragment thereof that binds to B-cell maturation antigen (BCMA), conjugated to a nucleic acid cross-linking agent; and (b) a proteasome inhibitor.

The content of the electronically submitted sequence listing in ASCII text file (Name: BCMA-150-US-PSP-SequenceListing.txt; Size: 11,279 bytes; and Date of Creation: May 28, 2019) filed with the application is incorporated herein by reference in its entirety.

### BACKGROUND OF THE INVENTION

Blood cancer is a term that is used to describe many different types of cancer that affect blood cells, bone marrow or the lymphatic system. It has been reported that blood cancers represent almost 10% of new cancer cases each year in the US, with greater than 1.2 million people living with or in remission from a blood cancer in the US alone. It is the fifth most common cancer in the UK, with more than 240,000 people living with blood cancer in the UK and 40,000 people being diagnosed with blood cancer each year. The three main groups are leukaemia, lymphoma, and myeloma, each of which represent a B-cell malignancy.

For example, the B-cell malignancy myeloma (e.g. multiple myeloma (MM)) is a malignancy of clonal plasma cells (e.g. B-cells) with ongoing DNA damage associated with progression from pre-malignant monoclonal gammopathy of undetermined significance (MGUS) to active MM. Current treatment regimens for myeloma include conventional corticosteroids, alkylating agents, proteasome inhibitors (PIs), and immunomodulatory drugs (IMiDs), which have helped to increase the overall survival rate of myeloma patients. A particularly interesting therapeutic avenue that has been explored in recent years has been immunotherapy (e.g. with monoclonal antibodies). For example, WO 2010/104949 and WO 2019/025983 describe antibodies which bind to an antigen known as B-cell maturation antigen (BCMA), which has been shown to have good selectivity for B-cell malignancies (in particular myeloma cells), with the described antibodies showing anti-B-cell malignancy activity.

Despite the increased availability of different treatments, the development of drug resistance underlies relapse of disease (particularly in myeloma), and the effectiveness of any treatment is limited by the maximum efficacy achievable by a tolerable dose.

There therefore exists a need for an improved medicament having anti-B-cell malignancy activity. The present invention addresses one or more of the above-mentioned problems.

### BRIEF SUMMARY OF THE INVENTION

The present invention relates to combination therapy for a B-cell malignancy.

The present invention is predicated on the surprising finding that a proteasome inhibitor (e.g. bortezomib) can be used to work in synergy with an anti-BCMA antibody-drug conjugate (and vice versa), increasing cytotoxicity of cells of a B-cell malignancy following contact with a combination of these agents. Thus, a seminal finding of the present invention is that a proteasome inhibitor can be employed to enhance the anti-B-cell malignancy activity of an antibody-drug conjugate (and vice versa), more particularly where the drug (or said antibody-drug conjugate) is a nucleic acid cross-linking agent.

In one aspect, provided herein is a B-cell malignancy medicament, comprising:
a. an antibody-drug conjugate (ADC) comprising an antibody or antigen-binding fragment thereof that binds to B-cell maturation antigen (BCMA), conjugated to a nucleic acid cross-linking agent; and
b. a proteasome inhibitor;
wherein the medicament provides an enhanced suppression of a B-cell malignancy when compared with an otherwise identical medicament lacking said proteasome inhibitor; or
wherein the medicament provides an enhanced suppression of a B-cell malignancy when compared with an otherwise identical medicament lacking said ADC.

In another aspect, provided herein is a therapeutic combination for use in treating a B-cell malignancy, said therapeutic combination comprising:
a. an ADC comprising an antibody or antigen-binding fragment thereof that binds to BCMA, conjugated to a nucleic acid cross-linking agent; and
b. a proteasome inhibitor;
wherein the therapeutic combination provides an enhanced suppression of a B-cell malignancy when compared with an otherwise identical composition lacking said proteasome inhibitor; or
wherein the therapeutic combination provides an enhanced suppression of a B-cell malignancy when compared with an otherwise identical composition lacking said ADC.

In another aspect, provided herein is a method for treating a B-cell malignancy, the method comprising administering to a subject a therapeutic combination comprising:
a. an ADC comprising an antibody or antigen-binding fragment thereof that binds to BCMA, conjugated to a nucleic acid cross-linking agent; and
b. a proteasome inhibitor;
wherein the therapeutic combination provides an enhanced suppression of a B-cell malignancy when compared with an otherwise identical composition lacking said proteasome inhibitor; or
wherein the therapeutic combination provides an enhanced suppression of a B-cell malignancy when compared with an otherwise identical composition lacking said ADC.

In another aspect, provided herein is an in vitro method for enhancing ADC suppression of a malignant B-cell, said method comprising contacting a malignant B-cell with (a) an ADC comprising an antibody or antigen-binding fragment thereof that binds to BCMA, conjugated to a nucleic acid cross-linking agent, in combination with (b) a proteasome inhibitor.

In another aspect, provided herein is an in vitro method for enhancing proteasome inhibitor suppression of a malignant B-cell, said method comprising contacting a malignant B-cell with (a) a proteasome inhibitor, in combination with (b) an ADC comprising an antibody or antigen-binding fragment thereof that binds to BCMA, conjugated to a nucleic acid cross-linking agent.

### BRIEF DESCRIPTION OF THE DRAWINGS

FIG. 1 shows that M2 is more cytotoxic against MM cells than its MMAF ADC homolog (M3). A Serial dilutions of M2 or M3 were added into MM cell cultures for 3d, followed by CCK8 cell viability assay. Shown are ED₅₀ values of M2 (black circle) and M3 (open circle) determined from one representative experiment from three repeats, with triplicates for each dose. MM1S(R) and H929(R) cells derived from MM1S and H929, respectively, are resistant to lenalidomide and pomalidomide. RPMI-BCMA, RPMI8226 cells overexpressing BCMA; RPMI, RPMI8226 cells. B M2 (black circle) or M3 (open circle) were added for 3d, followed by cell proliferation assay using [H³] thymidine incorporation (left) and viability assay based on CCK8 for RPM18226 and luminescent-based Cell-Titer Growth (CTG) for paired ANBL6 and ANBL6-BR (bortezomib (btz)-resistant) cells. C Dexamethasone (Dex)- and btz-resistant MM cell pairs were treated with M2 or M3 for 2d, followed by flow cytometry (FCM) analysis to determine percentages of apoptotic cells (Annexin V+/Aqua− and Annexin V+/Aqua+). ***, p<0.0005; **, p<0.005.

FIG. 2 shows that M2, more potently than M3, blocks BMSC-induced MM cell viability and is cytotoxic to primary patient-derived MM cells. A MM1Sluc cells, alone or with BMSCs, were treated with M2 or M3 for 4d, and cell viability was determined by BLI. B CFSE-labeled IMiDs-resistant MM1S(R) or H929(R) cells were incubated with indicated drugs in the presence or absence of BMSCs for 2d, followed by FCM analysis using Annexin V and Live/dead Aqua staining. Shown are percentages of Annexin V−/Aqua− (viable) CFSE+ MM cells from one of three experiments, with triplicates at each dose. C H929 cells, alone or with IL-6 (5 ng/ml), were treated with M2 for 2d, and the percentages of Annexin V−/Aqua− (viable) cells were measured. D CD138+ cells from a representative RRMM patient were incubated with M2 or M3 for 3d, and live/dead cell fractions were measured. E CD138+ cells from RRMM patients (n=3) were incubated with M2 for 3d, followed by CTG assay. F BMMCs of MM patients (NDMM=4, RRMM=2) were treated with M2 (10 μg/ml) for 5d. Percentages of viable CD38highCD138+ cells were determined by FCM analysis.

FIG. 3 shows that M2, more potently than M3, blocks cell proliferation and induces apoptosis in MM cell lines regardless of drug sensitivity. A M1 (isotype-PBD), M2 (anti-BCMA-PBD), M3 (anti-BCMA-MMAF homolog), and M4 (isotype-MMAF) were added to MM cells in triplicate for 3d, followed by CCK8 viability assay. B M2 or M3 were added for 3d, followed by proliferation assay using [H³]-thymidine incorporation. C ANBL6 (btz-sensitive) and ANBL6-BR (btz-resistant) cells were confirmed following 2d treatment with btz using CTG-based survival assay (left) and FCM-based apoptosis assay using Annexin V and Live/dead Aqua staining (right). D MM1S (upper panel) and MM1R (lower panel) cells were treated with indicated drugs. The percentages of Annexin V+ MM cells are shown.

FIG. 4 shows that M2 induces specific cytotoxicity against MM cells protected by BMSC and IL-6, and further depletes CD38highCD138+ patient MM cells. A Various drug sensitive and —resistant MM cell lines (n=6), alone or with BMSCs, were treated with M2 for 3d, followed by CTG assay. B BCMA-negative BMSC, PBMC, and NK cells were treated with M2 for 5d. C H929 cells, alone or with IL-6 (5 ng/ml), were treated with M2 for 3d. D BMMCs of a representative NDMM patient were incubated with M2 for 5d. M2 decreased CD38highCD138+ MM cells in a dose-dependent manner.

FIG. 5 shows that M2 together with bortezomib synergistically induces MM cell death. A-B MM cells were treated with indicated drugs for 2d, followed by FCM analysis using PI and Annexin V staining. Shown are results of one representative sample of each cell line (A) and summaries of percentages of Annexin V+ cells from three repeats (B). *, p<0.01; **, p<0.005, ***, p<0.002, C Data from CTG-based cell viability assays are used to determine combination index (CI). “Effect” means degree of reduction in cell viability by M2 and btz. CI<1 indicates synergism of both drugs. Similar results were obtained from additional 3 repeats. FIG. 6 shows that combination of low doses of M2 and btz trigger synergistic MM cell death. Indicated MM cell lines were incubated with M2 and btz for 3d, alone or together, followed by CTG-based viability assay. “Effect” represents fraction of cells showing decrease in viability with combined treatment of M2 plus btz treatment. Combination index (CI) of <1 indicates synergy. All experiments were performed in triplicate, and mean value is shown.

FIG. 7 shows that M2 combined with bortezomib induces more potent in vivo anti-MM activity and prolonged survival in mice, when compared with individual drug alone. A CB17 SCID mice (n=7 each group) with palpable MM1S tumour implants were randomized and treated with control vehicle, a single dose of M2 (0.4 mg/kg), six doses of btz (0.4 mg/kg), or combination of M2 and btz (M2+btz) for 2 weeks. Tumour growth was significantly inhibited in the combination-treated group compared with controls (cnt). (M2 vs M2+ btz, p=0.035; btz vs M2+btz, p<0.005; cnt vs M2+btz; p=0.0012; btz vs cnt, p<0.005; M2 vs cnt, p<0.005). *, p<0.04, **, p<0.005, B Body weights of the animals were followed. C Using Kaplan-Meier and log-rank analysis, the median overall survival of animals treated with combination therapy was significantly prolonged. (cnt, 22d; M2, 40.5d; btz, 35d; M2+btz, 57d) (M2 vs M2+btz, p<0.045; btz vs M2+btz, p<0.023; cnt vs M2+btz, p<0.002). D Tumour tissue sections from each group were immunohistochemically analyzed for Ki-67 (original magnification, ×400). FIG. 8 shows that combined treatments with M2 and btz significantly decreased in vivo growth of MM1 S xenografts, demonstrating the in vivo synergism of M2 and btz in treating myeloma. Tumours were removed at the same treatment day from representative mice.

FIG. 9 shows that M2 significantly induces phosphorylation of DNA damage response (DDR) signalling pathways in MM cells, regardless of p53 status and drug resistance. MM cells with wild type (MM1S, MM1R, H929) and mutated p53 (*) were treated with indicated doses of M2 (a-d) for indicated time periods (a), overnight (b, d), or 2d (c). Cell lysates were prepared and analysed by immunoblotting using specific antibodies for indicated molecules. cPARP, cleaved PARP; cCas3, cleaved caspase 3. Experiments were repeated three times.

FIG. 10 shows M2 significantly induces DDR signaling cascades followed by apoptosis in a BCMA-dependent manner. Indicated MM cells were treated with indicated doses of M2 (A-B, D-F) or M3 (A, F, G) for overnight (A, D, G) or 2d (B, E-F). Cell lysates were prepared and analyzed by immunoblotting using specific antibodies for indicated molecules. M2 but not M3 induces DDR signaling pathways. cPARP, cleaved PARP; cCas3, cleaved caspase 3. (C) BCMA levels were measured using qRT-PCR. BCMA^{medium}, BCMA^{low}, and BCMA^{high} were derived from the parental RPM18226.

FIG. 11 M2 treatment induces DDR-related gene expression including RAD51. RNA from viable H929 MM cells treated with M2 under sub-lethal conditions were analysed using the TagMan® human DNA Repair pathway array (a). Transcripts were normalized by geomean of internal controls, and fold changes in M2-treated relative to control (cnt) groups are shown. Cell lysates were made from various M2-treated MM cell lines for immunoblotting to determine protein levels of RAD51 (b-c).

### DETAILED DESCRIPTION OF THE INVENTION

In one aspect, provided herein is a B-cell malignancy medicament, comprising:
a. an antibody-drug conjugate (ADC) comprising an antibody or antigen-binding fragment thereof that binds to B-cell maturation antigen (BCMA), conjugated to a nucleic acid cross-linking agent; and
b. a proteasome inhibitor;
wherein the medicament provides an enhanced suppression of a B-cell malignancy when compared with an otherwise identical medicament lacking said proteasome inhibitor; or
wherein the medicament provides an enhanced suppression of a B-cell malignancy when compared with an otherwise identical medicament lacking said ADC.

Another aspect provides a therapeutic combination for use in treating a B-cell malignancy, said therapeutic combination comprising:
a. an ADC comprising an antibody or antigen-binding fragment thereof that binds to BCMA, conjugated to a nucleic acid cross-linking agent; and
b. a proteasome inhibitor;
wherein the therapeutic combination provides an enhanced suppression of a B-cell malignancy when compared with an otherwise identical composition lacking said proteasome inhibitor; or
wherein the therapeutic combination provides an enhanced suppression of a B-cell malignancy when compared with an otherwise identical composition lacking said ADC.

In a related aspect, there is provided a method for treating a B-cell malignancy, the method comprising administering to a subject a therapeutic combination comprising:
a. an ADC comprising an antibody or antigen-binding fragment thereof that binds to BCMA, conjugated to a nucleic acid cross-linking agent; and
b. a proteasome inhibitor;
wherein the therapeutic combination provides an enhanced suppression of a B-cell malignancy when compared with an otherwise identical composition lacking said proteasome inhibitor; or
wherein the therapeutic combination provides an enhanced suppression of a B-cell malignancy when compared with an otherwise identical composition lacking said ADC.

Said enhanced suppression of a B-cell malignancy may comprise one or more selected from an enhanced delay in tumour growth, an enhanced reduction in tumour size, an enhanced reduction in tumour metastasis, an enhanced survival rate in a subject comprising a B-cell malignancy, or a combination thereof.

The term “B-cell malignancy” embraces any disease in which B-cells become cancerous and divide without control (e.g. in the bone marrow and blood), and can invade other sites (e.g. tissues and lymph systems). In one embodiment, said B-cell malignancy is one or more selected from B-cell lymphoma, B-cell leukemia, myeloma (e.g. multiple myeloma and or myeloma progenitor cells), or a combination thereof. The term “B-cell” embraces both mature (differentiated) B-cells and precursors thereof (e.g. stem cells). For example, myeloma stem cells and myeloma progenitor cells are embraced.

In one embodiment, the B-cell malignancy is characterised by comprising a malignant B-cell which expresses BCMA. In one embodiment, said malignant B-cell expresses a high level of BCMA antigen (relative to a reference non-malignant B-cell). A malignant B-cell is considered to express a “high level of BCMA” when a level of BCMA antigen expression in the malignant B-cell is increased to a statistically significant level when compared to a level of BCMA expression in a non-malignant (e.g. healthy) B-cell.

Examples of a B-cell lymphoma include diffuse large B-cell lymphoma (DLBCL), follicular lymphoma, chronic lymphocytic leukemia (CLL), small lymphocytic lymphoma (SLL), mantle cell lymphoma (MCL), marginal zone lymphomas, burkitt lymphoma, lymphoplasmacytic lymphoma, primary central nervous system (CNS) lymphoma, and primary intraocular lymphoma. Examples of B-cell leukemia include B-cell chronic lymphocytic leukemia/small lymphocytic lymphoma, acute lymphoblastic leukemia, B-cell prolymphocytic leukemia, Precursor B lymphoblastic leukemia, and hairy cell leukemia.

In one embodiment, said B-cell malignancy is myeloma (e.g. multiple myeloma).

Multiple myeloma (MM), also known as plasma cell myeloma or Kahler's disease, is a cancer of B-cells (plasma cells), which are a type of white blood cell normally responsible for the production of antibodies. Current therapies for MM include chemotherapy, radiation, surgery, biophosphonates, and autologous stem-cell transplantation (ASCT). While these therapies often cause remissions, nearly all patients eventually relapse and die. Multiple myeloma affects 1-4 per 100,000 people per year. The disease is more common in men, and for yet unknown reasons is twice as common in African Americans as it is in Caucasian Americans.

B-cell maturation antigen (BCMA), also known as tumour necrosis factor receptor superfamily member 17 (TNFRSF17), is a tumour necrosis family receptor (TNFR) member expressed on cells of the B-cell lineage. BCMA expression is highest on terminally differentiated B-cells. BCMA is involved in mediating the survival of plasma cells for maintaining long-term humoral immunity. The expression of BCMA has been linked to a number of cancers, autoimmune disorders, and infectious diseases. BCMA RNA has been detected universally in multiple myeloma cells, and BCMA protein has been detected on the surface of plasma cells from multiple myeloma patients by several investigators. As such, BCMA represents a therapeutic target for B-cell malignancies, particularly multiple myeloma.

The nucleotide sequence of human BCMA (TNFRSF17) is described in Ensemble (see accession number ENSG00000048462), which is incorporated herein by reference. The amino acid sequence of BCMA (TNFRSF17) is described in UniProt (see accession number Q02223, which is incorporated herein by reference). The amino acid sequence of human BCMA is provided in SEQ ID NO: 13.

BCMA is also expressed on multiple myeloma stem cells. As such, the term “myeloma” embraces multiple myeloma “stem” cells, and myeloma “progenitor” cells. Furthermore, methods and uses of the invention embrace treating malignancies comprising multiple myeloma stem cells (e.g. that express BCMA). Multiple myeloma stem cells (and/or myeloma progenitor cells) can be identified in the bone marrow of multiple myeloma patients by their surface expression of CD19 and lack of CD138 surface expression. These cells are uniquely clonogenic and engraft immunodeficient mice, whereas the myeloma plasma cells, defined as CD138+CD19−, do not.

The term “antibody-drug conjugate” means an antibody (or antigen-binding fragment thereof) attached to a cytotoxic agent (generally a small molecule drug with a high systemic toxicity) via chemical linkers. This term is used herein to describe an antibody, or antigen-binding fragment thereof, that is conjugated to a nucleic acid cross-linking agent. In one embodiment, an ADC may comprise a nucleic acid cross-linking agent (e.g. a small molecule cytotoxin) that has been chemically modified to contain a linker. The linker may then be used to conjugate the nucleic acid cross-linking agent (cytotoxin) to the antibody or to the antigen-binding fragment thereof. Upon binding to the target antigen on the surface of a cell (e.g. BCMA), the ADC is internalized and trafficked to the lysosome where the nucleic acid cross-linking agent (cytotoxin) is released by either proteolysis of a cleavable linker (e.g., by cathepsin B found in the lysosome) or by proteolytic degradation of the antibody, e.g. if attached to the cytotoxin via a non-cleavable linker. The cytotoxin then translocates out of the lysosome and into the cytosol or nucleus, where it can then bind to its target, depending on its mechanism of action.

In one embodiment, said nucleic acid cross-linking agent is a cytotoxic nucleic acid cross-linking agent.

Proteasome inhibitors have found utility in a number of cancer therapies. The inventors have surprisingly found that a proteasome inhibitor may be employed to enhance (e.g. synergistically enhance) the anti-B-cell malignancy activity of an ADC of the invention, and conversely, an ADC of the invention may be employed to enhance (e.g. synergistically enhance) the anti-B-cell malignancy activity of a proteasome inhibitor. Without wishing to be bound by theory, the present inventors believe that the activity of a proteasome inhibitor may enhance the activity of an ADC of the invention by causing a downstream (molecular) effect leading to the suppression of molecule(s), e.g. which may normally inhibit the activity of the nucleic acid cross-linking agent (e.g. PBD cytotoxin) of the ADC, and that may even lead to resistance to the nucleic acid cross-linking agent. This activity may be linked to or be separated from its ‘normal’ activity as a direct proteasome inhibitor. This theory is supported by the observation that a therapeutic combination of the invention shows enhanced activity even against malignant B-cells which are resistant to a proteasome inhibitor (e.g. bortezomib) as a monotherapy.

Conversely, without wishing to be bound by theory, the activity of an ADC of the invention may enhance the activity of a proteasome inhibitor by causing a downstream (molecular) effect leading to the suppression of molecule(s), e.g. which may normally inhibit the activity of the proteasome inhibitor, and that may even lead to resistance to the proteasome inhibitor.

Proteasome inhibitors generate several consequences. For example, they may cause increased levels of biologically active proteins, such as IKB, which is the inhibitor of nuclear factor-kappaB (a protein involved in cell survival). In addition, misfolded and other obsolete proteins accumulate as well and trigger the unfolded protein response (UPR), which entails endoplasmic reticulum (ER) stress.

In one embodiment, the proteasome inhibitor is a boronic-acid based proteasome inhibitor (e.g. bortezomib).

In one embodiment, the proteasome inhibitor is one or more selected from bortezomib, carfilzomib, ixazomib, marizomib, oprozomib, delanzomib, or a combination thereof. In one embodiment, the proteasome inhibitor is bortezomib.

Bortezomib (e.g. Velcade), formerly known as PS-341 (Millennium Pharmaceuticals, Cambridge, Mass., USA) functions as an inhibitor of the 26S proteasome, a multisubunit protein complex that is responsible for the degradation of ubiquitinated proteins. Bortezomib is a peptide boronate with the molecular formula C₁₉H₂₅BN₄O:

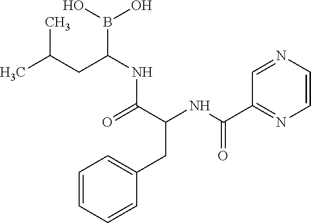 [SMILES: CC(C)CC(NC(=O)C(Cc1ccccc1)NC(=O)c1cnccn1)B(O)O]

Carfilzomib (Kyprolis®), is an epoxyketone Proteasome Inhibitor binding irreversibly to the β 5 subunit (PSMB5). Its formula is:

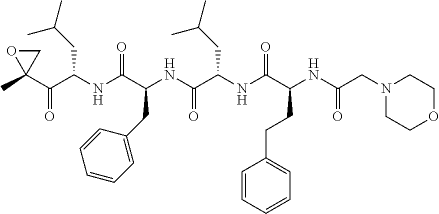 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

Ixazomib (Ninlaro®) selectively and reversibly inhibits the protein proteasome subunit beta type-5 (PSMB5). Its formula is:

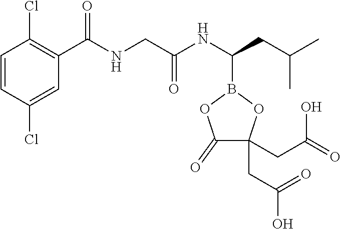 [SMILES: CC(C)C[C@H](NC(=O)CNC(=O)c1cc(Cl)ccc1Cl)B1OC(=O)C(CC(=O)O)(CC(=O)O)O1]

Marizomib (Salinosporamide A) inhibits proteasome activity by covalently modifying the active site threonine residues of the 20S proteasome. It binds to 3 major catalytic sites on protein proteasome subunits β5, β1 and β2, irreversibly. Its formula is:

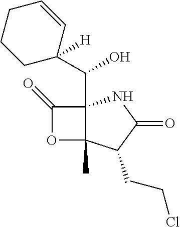

Oprozomib (known as ONX 0912) has the following formula:

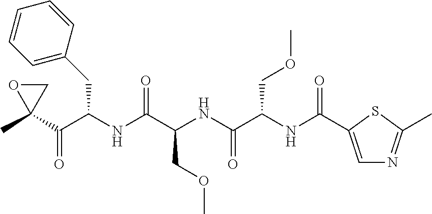

Delanzomib (known as CEP-18 770) has the following formula:

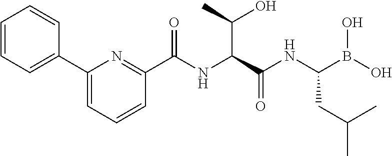 [SMILES: CC(C)C[C@H](NC(=O)[C@@H](NC(=O)c1cccc(-c2ccccc2)n1)[C@@H](C)O)B(O)O]

In one embodiment, the medicament and/or the therapeutic composition is comprised within a pharmaceutical composition. The term “pharmaceutical composition” refers to a preparation that is in such form as to permit the biological activity of the active ingredient to be effective, and which contains no additional components which are unacceptably toxic to a subject to which the composition would be administered. Such composition can be sterile. The medicament and/or the therapeutic composition may comprise a pharmaceutically acceptable carrier. An example carrier is physiological saline. Suitable pharmaceutical compositions can comprise one or more of a buffer (e.g., acetate, phosphate or citrate buffer), a surfactant (e.g., polysorbate), a stabilizing agent (e.g., human albumin), a preservative (e.g., benzyl alcohol), and absorption promoter to enhance bioavailability, and/or other conventional solubilizing or dispersing agents.

In one embodiment, a medicament, therapeutic combination or pharmaceutical composition of the invention can comprise a pharmaceutically acceptable, non-toxic, sterile carrier such as physiological saline, non-toxic buffers, preservatives and the like. Suitable formulations for use in the therapeutic methods disclosed herein are described in Remington's Pharmaceutical Sciences, 22nd ed., Ed. Lloyd V. Allen, Jr. (2012). In one embodiment, a medicament, therapeutic combination or pharmaceutical composition of the invention may be comprised within one or more formulation selected from a capsule, a tablet, an aqueous suspension, a solution, a nasal aerosol, or a combination thereof. In one embodiment, a pharmaceutical composition may comprise a buffer (e.g., acetate, phosphate or citrate buffer), a surfactant (e.g., polysorbate), optionally a stabilizer agent (e.g., human albumin), etc.

An agent which is “cytotoxic” (referred to herein as a “cytotoxin” or “cytotoxic agent”) is an agent that inhibits or prevents the function of cells and/or causes destruction of cells (cell death), and/or exerts anti-proliferative effects. It will be appreciated that a cytotoxin or cytotoxic agent of an ADC is also referred to in the art as the “payload” of the ADC.

The term “nucleic acid cross-linking agent” means a molecule which reacts with two nucleotides of a nucleic acid, forming a covalent linkage between them. This crosslink can occur within the same strand (intrastrand) or between opposite strands of double-stranded DNA (interstrand). These links (adducts) interfere with cellular metabolism, such as DNA replication and transcription, typically triggering cell death. In one embodiment, the nucleic acid is DNA.

In one embodiment, the nucleic acid cross-linking agent is an agent which damages DNA by inducing DNA strand breakage (single strand break and/or double strand break), and typically subsequently leading to apoptosis. In one embodiment, the nucleic acid cross-linking agent is a cytotoxic nucleic acid cross-linking agent.

In one embodiment, the nucleic acid cross-linking agent is one or more selected from a pyrrolobenzodiazepine (PBD), a nitrogen mustard, cisplatin, a chloro ethyl nitroso urea

(CENU), a psoralen, a mitomycin C (MMC) antibiotic, or a combination thereof. Said nitrogen mustard may be one or more selected from cyclophosphamide, chlormethine (e.g. mechlorethamine or mustine), uramustine, uracil mustard, melphalan, chlorambucil, ifosfamide, bendamustine or a combination thereof. In one embodiment, said CENU is carmustine.

In one embodiment, the nucleic acid cross-linking agent is a pyrrolobenzodiazepine (PBD).

The term “pyrrolobenzodiazepine” embraces both a pyrrolobenzodiazepine as well as a functional derivative thereof.

PBDs are a class of cytotoxic agents which translocate to the nucleus before crosslinking DNA, preventing replication during mitosis, damaging DNA by inducing DNA strand breakage (single strand break and/or double strand break), and subsequently leading to apoptosis. Some PBDs also have the ability to recognize and bind to specific sequences of DNA. In one embodiment, the PBD comprises the general structure:

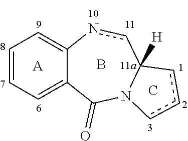

PBDs differ in the number, type, and position of substituents, in both their aromatic A rings and pyrrolo C rings, and in the degree of saturation of the C ring. In the B-ring there is either an imine (N═C), a carbinolamine (NH—CH(OH)), or a carbinolamine methyl ether (NH—CH(OMe)) at the N10-C11 position, which is the electrophilic centre responsible for alkylating DNA. All of the known natural products have an (S)-configuration at the chiral C11a position which provides them with a right-handed twist when viewed from the C ring towards the A ring. This feature also gives PBDs the appropriate three-dimensional shape for isohelicity with the minor groove of B-form DNA, leading to a snug fit at the binding site. PBDs can form adducts in the minor groove, leading to interference with DNA processing.

The first PBD anti-tumour antibiotic, anthramycin, was discovered in 1965. Since then, a number of naturally occurring PBDs have been reported, and over 10 synthetic routes have been developed to a variety of analogues. Family members include abbeymycin, chicamycin, DC-81, mazethramycin, neothramycins A and B, porothramycin, prothracarcin, sibanomicin (DC-102), sibiromycin and tomamycin. PBDs and ADCs comprising them are also described in WO 2015/155345 and WO 2015/157592, incorporated in their entirety herein by reference.

In one embodiment, the PBD is PBD 3249, also referred to herein as “SG3249” (e.g described in more detail in WO 2014/057074, incorporated herein by reference). PBD 3249 (SG3249) comprises the following structure:

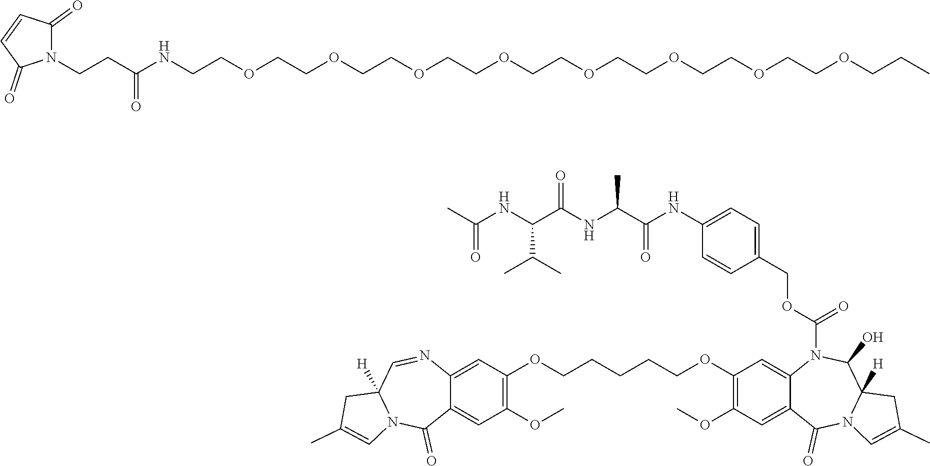

In one embodiment, the PBD is PBD 3315, also referred to herein as “SG3315” (e.g. described in more detail in WO 2015/052322, incorporated herein by reference). PBD 3315 (SG3315) comprises the following structure:

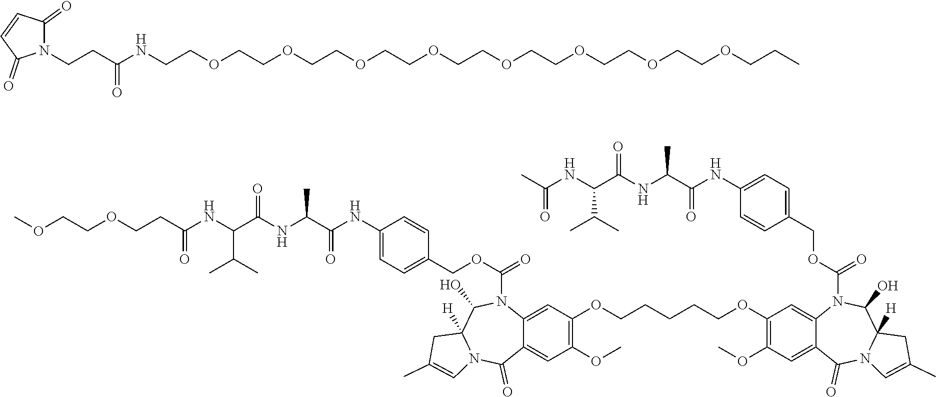

In one embodiment, the PBD (e.g. described in detail in WO 2017/137553, incorporated herein by reference) comprises the formula:

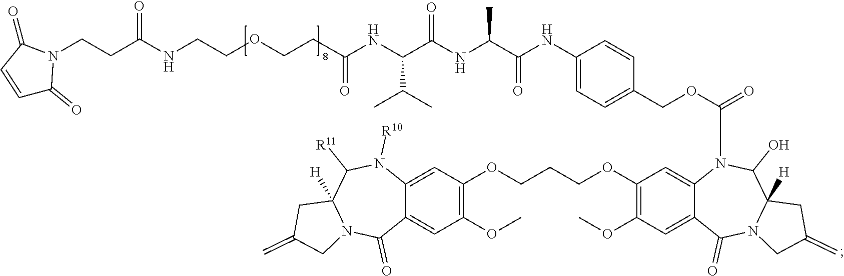

wherein either:
(a) R¹⁰ and R¹¹ form a nitrogen-carbon double bond between the nitrogen and carbon atoms to which they are bound; or
(b) R¹⁰ is OH, and R¹¹ is:

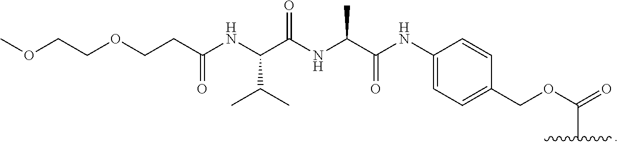 [SMILES: *C(=O)OCc1ccc(NC(=O)[C@H](C)NC(=O)[C@@H](NC(=O)CCOCCOC)C(C)C)cc1]

In another embodiment, the PBD is SG3400, also referred to as Compound 23 (e.g. described in detail in WO 2017/137553, incorporated herein by reference) and has the following structure:

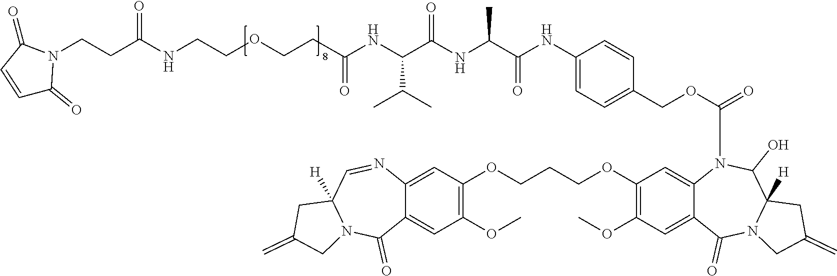

In one embodiment, the PBD is a PBD dimer comprising at least two PBD monomers. For instance, the at least two PBD monomers are linked through their aromatic A-ring phenolic C8-positions via a flexible propyldioxy tether.

The antibody or antigen binding fragment thereof of the invention may be conjugated to a cytotoxin (a heterologous agent) such as PBD using site-specific or non-site specific methods of conjugation. In one embodiment, the antibody or antigen binding fragment thereof comprises one, two, three, four or more PBD moieties. In one embodiment, all PBD moieties (conjugated to the antibody or antigen fragment thereof) comprise the same structure.

A nucleic acid cross-linking agent (a cytotoxin) of the invention may be linked (e.g. conjugated) to the antibody or antigen binding fragment thereof by means of a spacer (e.g. at least one spacer). In one embodiment, the spacer is a peptide spacer. In one embodiment, the spacer is a non-peptide (e.g. chemical) spacer.

Conventional conjugation strategies for antibodies or antigen-binding fragments thereof rely on randomly conjugating the payload (cytotoxin) to the antibody or antigen binding fragment through lysines or cysteines. In one embodiment, the antibody or antigen-binding fragment thereof is randomly conjugated to an agent (e.g. cytotoxin), for example, by partial reduction of the antibody or fragment, followed by reaction with a desired agent, with or without a linker moiety attached. The antibody or antigen binding fragment may be reduced using DTT or similar reducing agent. The agent with or without a linker moiety attached can then be added at a molar excess to the reduced antibody or fragment in the presence of DMSO. After conjugation, excess free cysteine may be added to quench unreacted agent. The reaction mixture may then be purified and buffer-exchanged into PBS.

In one embodiment, a nucleic acid cross-linking agent (e.g. a cytotoxin) is conjugated to an antibody or antigen binding fragment thereof by site-specific conjugation. In one embodiment, site-specific conjugation of a nucleic acid cross-linking agent (e.g. a cytotoxin) to an antibody or antigen binding fragment thereof using reactive amino acid residues at specific positions yields a homogeneous ADC preparation with uniform stoichiometry.

The site specific conjugation can be through a cysteine residue or a non-natural amino acid. In one embodiment, the nucleic acid cross-linking agent (e.g. cytotoxin) is conjugated to the antibody or antigen binding fragment thereof through at least one cysteine residue.

In one embodiment, the nucleic acid cross-linking agent (e.g. cytotoxin) is chemically conjugated to the side chain of an amino acid (for example, at a specific Kabat position in an Fc region of the antibody or antigen binding fragment). In one embodiment, the nucleic acid cross-linking agent (e.g. cytotoxin) is conjugated to the antibody or antigen binding fragment thereof through a cysteine substitution at any suitable position of the Fc region, through a cysteine of at least one of positions 239, 248, 254, 273, 279, 282, 284, 286, 287, 289, 297, 298, 312, 324, 326, 330, 335, 337, 339, 350, 355, 356, 359, 360, 361, 375, 383, 384, 389, 398, 400, 413, 415, 418, 422, 440, 441, 442, 443 and 446, wherein the numbering corresponds to the EU index in Kabat. In one embodiment, the specific positions are 239, 442, or both, wherein the numbering corresponds to the EU index in Kabat. In one embodiment, the specific positions are position 442, an amino acid (cysteine) insertion between positions 239 and 240, or both, wherein the numbering corresponds to the EU index in Kabat. In one embodiment, the nucleic acid cross-linking agent (cytotoxin) is conjugated to the antibody or antigen binding fragment thereof through a thiol-maleimide linkage. In some aspects, the amino acid side chain is a sulfhydryl side chain, e.g. a sulfhydryl reactive group located at the hinge and heavy-light chains of the antibody or antigen-binding fragment thereof.

In one embodiment, the antibody or antigen binding fragment thereof comprises a heavy chain constant region comprising the amino acid sequence of SEQ ID NO: 11.

In one embodiment, the antibody or antigen binding fragment thereof comprises a human kappa constant region comprising the amino acid sequence of SEQ ID NO: 12.

In one embodiment, the antibody or antigen binding fragment thereof comprises:
i. a HCDR1 comprising the amino acid sequence of SEQ ID NO: 1, or a functional variant thereof;
ii. a HCDR2 comprising the amino acid sequence of SEQ ID NO: 2, or a functional variant thereof;
iii. a HCDR3 comprising the amino acid sequence of SEQ ID NO: 3, or a functional variant thereof;
iv. a LCDR1 comprising the amino acid sequence of SEQ ID NO: 4, or a functional variant thereof;
v. a LCDR2 comprising the amino acid sequence of SEQ ID NO: 5, or a functional variant thereof; and
vi. a LCDR3 comprising the amino acid sequence of SEQ ID NO: 6, or a functional variant thereof.

In one embodiment, the antibody or antigen binding fragment thereof comprises:
i. a variable heavy chain (VH) comprising an amino acid sequence having at least 70%, 75%, 80%, 90%, or 95% sequence identity to a reference amino acid sequence of SEQ ID NO: 7, or a functional variant thereof; and/or
ii. a variable light chain (VL) comprising an amino acid sequence having at least 70%, 75%, 80%, 90%, or 95% sequence identity to a reference amino acid sequence SEQ ID NO: 8, or a functional variant thereof.

In one embodiment, the antibody or antigen binding fragment thereof comprises a variable heavy chain comprising the amino acid sequence of SEQ ID NO: 7, or a functional variant thereof. In one embodiment, the antibody or antigen binding fragment thereof comprises a variable light chain comprising the amino acid sequence of SEQ ID NO: 8, or a functional variant thereof.

In one embodiment, the antibody or antigen binding fragment thereof comprises:
i. a variable heavy chain comprising the amino acid sequence of SEQ ID NO: 7, or a functional variant thereof; and
ii. a variable light chain comprising the amino acid sequence of SEQ ID NO: 8, or a functional variant thereof.

SEQ ID NO: 7 and SEQ ID NO: 8 are germlined versions of a VH and VL. Alternatively, an antibody or antigen binding fragment thereof may comprise a non-germlined VH and/or VL (e.g. a VH of SEQ ID NO: 9 and a VL of SEQ ID NO: 10).

The present invention encompasses the antibodies (e.g. the antibody or antigen binding fragment) defined herein having the recited CDR sequences or variable heavy and variable light chain sequences (reference antibodies), as well as functional variants thereof. A functional variant binds to the same target antigen as the reference antibody (e.g. BCMA), and may exhibit the same antigen cross-reactivity (or lack thereof) as the reference antibody. The functional variants may have a different affinity for the target antigen when compared to the reference antibody. In one embodiment, the functional variants have substantially the same affinity.

In one embodiment functional variants of a reference antibody show sequence variation at one or more CDRs when compared to corresponding reference CDR sequences. Thus, a functional antibody variant may comprise a functional variant of a CDR. Where the term “functional variant” is used in the context of a CDR sequence, this means that the CDR has at most 2, or at most 1 amino acid difference(s) when compared to a corresponding reference

CDR sequence, and when combined with the remaining 5 CDRs (or variants thereof) enables the variant antibody to bind to the same target antigen (e.g. BCMA) as the reference antibody. In one embodiment, the functional variant exhibits the same antigen cross-reactivity (or lack thereof) as the reference antibody.

In one embodiment a functional variant antibody or antigen binding fragment thereof comprises:
a light chain CDR1 having at most 2 amino acid difference(s) when compared to a corresponding reference CDR sequence;
a light chain CDR2 having at most 2 amino acid difference(s) when compared to a corresponding reference CDR sequence;
a light chain CDR3 having at most 2 amino acid difference(s) when compared to a corresponding reference CDR sequence;
a heavy chain CDR1 having at most 2 amino acid difference(s) when compared to a corresponding reference CDR sequence;
a heavy chain CDR2 having at most 2 amino acid difference(s) when compared to a corresponding reference CDR sequence; and
a heavy chain CDR3 having at most 2 amino acid difference(s) when compared to a corresponding reference CDR sequence;
wherein the functional variant binds to the same target antigen as the reference antibody. In one embodiment, the functional variant exhibits the same antigen cross-reactivity (or lack thereof) as the reference antibody.

In one embodiment, a functional variant antibody or antigen binding fragment thereof comprises:
a light chain CDR1 having at most 1 amino acid difference when compared to a corresponding reference CDR sequence;
a light chain CDR2 having at most 1 amino acid difference when compared to a corresponding reference CDR sequence;
a light chain CDR3 having at most 1 amino acid difference when compared to a corresponding reference CDR sequence;
a heavy chain CDR1 having at most 1 amino acid difference when compared to a corresponding reference CDR sequence;
a heavy chain CDR2 having at most 1 amino acid difference when compared to a corresponding reference CDR sequence; and
a heavy chain CDR3 having at most 1 amino acid difference when compared to a corresponding reference CDR sequence;
wherein the functional variant binds to the same target antigen as the reference antibody. In one embodiment, a functional variant exhibits the same antigen cross-reactivity (or lack thereof) as the reference antibody.

For example, a functional variant of the antibody or antigen binding fragment may comprise:
a heavy chain CDR1 having at most 2 amino acid difference(s) when compared to SEQ ID NO: 1;
a heavy chain CDR2 having at most 2 amino acid difference(s) when compared to SEQ ID NO: 2; and
a heavy chain CDR3 having at most 2 amino acid difference(s) when compared to SEQ ID NO: 3;
a light chain CDR1 having at most 2 amino acid difference(s) when compared to SEQ ID NO: 4;
a light chain CDR2 having at most 2 amino acid difference(s) when compared to SEQ ID NO: 5;
a light chain CDR3 having at most 2 amino acid difference(s) when compared to SEQ ID NO: 6;
wherein the variant antibody binds to BCMA (e.g. BCMA polypeptide epitope), and/or wherein the variant antibody may exhibit the same antigen cross-reactivity (or lack thereof) as the reference antibody or antigen binding fragment.

In one embodiment, a functional variant of the antibody or antigen binding fragment may comprise:
a heavy chain CDR1 having at most 1 amino acid difference when compared to SEQ ID NO: 1;
a heavy chain CDR2 having at most 1 amino acid difference when compared to SEQ ID NO: 2; and
a heavy chain CDR3 having at most 1 amino acid difference when compared to SEQ ID NO: 3;
a light chain CDR1 having at most 1 amino acid difference when compared to SEQ ID NO: 4;
a light chain CDR2 having at most 1 amino acid difference when compared to SEQ ID

NO: 5;
a light chain CDR3 having at most 1 amino acid difference when compared to SEQ ID NO: 6;
wherein the variant antibody binds to BCMA (e.g. BCMA polypeptide epitope), and/or wherein the variant antibody may exhibit the same antigen cross-reactivity (or lack thereof) as the reference antibody or antigen binding fragment.

The foregoing can be applied analogously to variants of the other antibodies described herein, wherein the amino acid differences are defined relative to the CDR sequences thereof, and wherein the variant antibody binds to the same target antigen as said antibodies, and/or wherein the variant antibody may exhibit the same antigen cross-reactivity (or lack thereof).

In one embodiment, a functional variant antibody may have at most 5, 4 or 3 amino acid differences total in the CDRs thereof when compared to a corresponding reference antibody, with the proviso that there is at most 2 (e.g. at most 1) amino acid differences per CDR. In one embodiment a functional variant antibody has at most 2 (e.g. at most 1) amino acid differences total in the CDRs thereof when compared to a corresponding reference antibody, with the proviso that there is at most 2 amino acid differences per CDR. In one embodiment, a functional variant antibody has at most 2 (e.g. at most 1) amino acid differences total in the CDRs thereof when compared to a corresponding reference antibody, with the proviso that there is at most 1 amino acid difference per CDR.

The amino acid difference may be an amino acid substitution, insertion or deletion. In one embodiment the amino acid difference is a conservative amino acid substitution as described herein.

In one embodiment a functional variant antibody has the same framework sequences as the exemplary antibodies described herein. In another embodiment the functional variant antibody may comprise a framework region having at most 2, or at most 1 amino acid difference (when compared to a corresponding reference framework sequence). Thus, each framework region may have at most 2, or at most 1 amino acid difference (when compared to a corresponding reference framework sequence).

In one embodiment a functional variant antibody may have at most 5, 4 or 3 amino acid differences total in the framework regions thereof when compared to a corresponding reference antibody, with the proviso that there is at most 2 (e.g. at most 1) amino acid differences per framework region. In one embodiment a functional variant antibody has at most 2 (e.g. at most 1) amino acid differences total in the framework regions thereof when compared to a corresponding reference antibody, with the proviso that there is at most 2 amino acid differences per framework region. In one embodiment a functional variant antibody has at most 2 (e.g. at most 1) amino acid differences total in the framework regions thereof when compared to a corresponding reference antibody, with the proviso that there is at most 1 amino acid difference per framework region.

Thus, a functional variant antibody may comprise a variable heavy chain and a variable light chain as described herein, wherein:
the heavy chain has at most 14 amino acid differences (at most 2 amino acid differences in each CDR and at most 2 amino acid differences in each framework region) when compared to a heavy chain sequence described herein (e.g. SEQ ID NO.: 7); and
the light chain has at most 14 amino acid differences (at most 2 amino acid differences in each CDR and at most 2 amino acid differences in each framework region) when compared to a light chain sequence described herein (e.g. SEQ ID NO.: 8);
wherein the functional variant antibody binds to the same target antigen as the reference antibody, and/or wherein the functional variant antibody exhibits the same antigen cross-reactivity (or lack thereof) as the reference antibody.

Said variant heavy or light chains may be referred to as “functional equivalents” of the reference heavy or light chains.

In one embodiment a functional variant antibody may comprise a variable heavy chain and a variable light chain as described herein, wherein:
the heavy chain has at most 7 amino acid differences (at most 1 amino acid difference in each CDR and at most 1 amino acid difference in each framework region) when compared to a heavy chain sequence herein (e.g. SEQ ID NO.: 7); and
the light chain has at most 7 amino acid differences (at most 1 amino acid difference in each CDR and at most 1 amino acid difference in each framework region) when compared to a light chain sequence herein (e.g. SEQ ID NO.: 8);
wherein the functional variant antibody binds to the same target antigen as the reference antibody, and/or wherein the functional variant antibody exhibits the same antigen cross-reactivity (or lack thereof) as the reference antibody.

In one embodiment, the antibody or antigen binding fragment thereof binds to BCMA (e.g. a human BCMA) with a dissociation constant (KD) of ≤1 μM, ≤100 nM, ≤10 nM, ≤1nM, ≤0.1 nM, ≤10 pM, ≤1 pM, or ≤0.1 pM. In one embodiment, the antibody or antigen binding fragment thereof binds to BCMA (e.g. a human BCMA) with a KD of between about 0.1 nM to about 40 nM, between about 0.5 nM to about 30 nM, between about 1 nM to about 20 nM, or between about 1.5 nM to about 20 nM. In one embodiment, the antibody or antigen binding fragment thereof binds to BCMA (e.g. a human BMCA) with a KD of between about 23 nM to about 27 nM. In one embodiment, the antibody or antigen binding fragment thereof binds to BCMA (e.g. a human BCMA) with a KD of between about 1 nM to about 1.5 nM. The KD measurements (binding affinity) may be carried out by any suitable assay known in the art. Such methods include, for example, fluorescence activated cell sorting (FACS), surface plasmon resonance (e.g., Biacore, ProteOn), biolayer interferometry (BLI, e.g. Octet), kinetics exclusion assay (e.g. KinExA), separable beads (e.g., magnetic beads), antigen panning, ELISA, and/or or ForteBio Octet system. Suitable kinetics exclusion assays include a KinExA system (e.g., KinExA 3100, KinExA 3200, or KinExA 4000) (Sapidyne Instruments, Idaho).

Advantageously, this therapeutic combination provides purposeful use of such proteasome inhibitors for targeting cells that would normally be unreactive (e.g. resistant) to such proteasome inhibitors. For example, the inventors have demonstrated that a proteasome inhibitor (e.g. bortezomib) enhances the anti-B-cell malignancy activity of an ADC even when the B-cell malignancy is resistant to the proteasome inhibitor (see Example 3).

Thus, the present invention embraces administering an ADC in combination with a proteasome inhibitor at a proteasome inhibitor dose that otherwise provides only a low/poor suppression of a B-cell malignancy, such that following said addition the combination is now capable of demonstrating an improved suppression of a B-cell malignancy. Therefore, the present invention provides a ‘re-purposing’ of a proteasome inhibitor (e.g. bortezomib) for use as an enhancing agent for an ADC, as opposed to use a (standalone) monotherapy (which would otherwise not be efficacious against resistant malignancies).

Similarly, the invention embraces administering a proteasome inhibitor in combination with an ADC at an ADC dose that otherwise provides only a low/poor suppression of a B-cell malignancy, such that following said addition the combination is now capable of demonstrating an improved suppression of a B-cell malignancy. Therefore, the prevent invention provides a ‘re-purposing’ of an ADC for use as an enhancing agent fora proteasome inhibitor, as opposed to use a (standalone) monotherapy (which would otherwise not be efficacious against a B-cell malignancy expressing a low level of BCMA antigen).

Thus, in one embodiment, the B-cell malignancy is resistant to a proteasome inhibitor (e.g. a medicament comprising a proteasome inhibitor in the absence of an ADC of the invention). In one embodiment, said proteasome inhibitor is bortezomib.

In one embodiment, the B-cell malignancy is resistant to an ADC of the invention (e.g. a medicament comprising an ADC in the absence of a proteasome inhibitor of the invention). In one embodiment, a B-cell malignancy that is resistant to an ADC of the invention is characterised by comprising a malignant B-cell having no increase or a decrease in an expression level of a BCMA antigen relative to a reference non-malignant B-cell.

The therapeutic combination has also been shown to have enhanced activity against a B-cell malignancy that is resistant to a number of other common anti-cancer drugs (see Example 3). Thus, in one embodiment, the B-cell malignancy is resistant to one or more drug selected from dexamethasone, lenalidomide, pomalidomide, bortezomib, or a combination thereof.

Furthermore, due to the synergistic nature of the combination, lower doses of the component parts may be employed, thus reducing the risk of the development of resistance (a fundamental public health threat) to either of the component parts due to overuse. Indeed, the present invention reduces the need for the prescription of chronic treatment regimens. For example, the inventors have shown that in vivo efficacy of sub-optimal doses of the ADC is still enhanced when administered in combination with a proteasome inhibitor (e.g. bortezomib).

Thus, in one embodiment the ADC and/or the proteasome inhibitor is administered at a sub-optimal dose.

The order of application/administration of the component parts of the therapeutic combination can be varied. The ADC and the proteasome inhibitor can be administered simultaneously (e.g. both at their own particular optimal dose for achieving synergy), either as part of a single composition or within separate compositions. For example, the ADC may be present in a first composition (e.g. adapted for intravenous administration to a subject) and the proteasome inhibitor may be present in a second composition (e.g. adapted for intravenous, subcutaneous or oral administration to a subject). For example, where the proteasome inhibitor is bortezomib, said second composition may be adapted for intravenous or subcutaneous injection. In embodiments in which the proteasome inhibitor is ixazomib, said second composition may additionally or alternatively be adapted for oral administration.

Furthermore, the ADC and the proteasome inhibitor may be administered at different times (e.g. a proteasome inhibitor may be pre-administered to sensitise a malignant B-cell to the ADC). Thus, in a further embodiment an ADC and a proteasome inhibitor are administered to a subject at different times, within separate compositions.

In one embodiment a proteasome inhibitor is administered prior to an ADC. In one embodiment a proteasome inhibitor is administered simultaneously with an ADC. In one embodiment a proteasome inhibitor is administered sequentially to an ADC.

The term “treat” or “treating” as used herein encompasses prophylactic treatment (e.g. to prevent onset of a B-cell malignancy) as well as corrective treatment (treatment of a subject already suffering from a B-cell malignancy). In one embodiment, the term “treat” or “treating” as used herein means corrective treatment. The term “treat” or “treating” encompasses treating both the B-cell malignancy and a symptom thereof. In some embodiments “treat” or “treating” refers to a symptom of a B-cell malignancy.

Therefore, a medicament and/or therapeutic combination may be administered to a subject in a therapeutically effective amount or a prophylactically effective amount.

A “therapeutically effective amount” is any amount of the medicament and/or therapeutic combination, which when administered alone or in combination to a subject for treating a B-cell malignancy (or a symptom thereof) is sufficient to effect such treatment of the B-cell malignancy, or symptom thereof.

A “prophylactically effective amount” is any amount of the medicament and/or therapeutic combination that, when administered alone or in combination to a subject inhibits or delays the onset or reoccurrence of a B-cell malignancy (or a symptom thereof). In some embodiments, the prophylactically effective amount prevents the onset or reoccurrence of a B-cell malignancy entirely. “Inhibiting” the onset means either lessening the likelihood of B-cell malignancy onset (or symptom thereof), or preventing the onset entirely.

A further aspect of the invention provides an in vitro method for enhancing ADC suppression of a malignant B-cell, said method comprising contacting a malignant B-cell with (a) an ADC comprising an antibody or antigen-binding fragment thereof that binds to BCMA, conjugated to a nucleic acid cross-linking agent, in combination with (b) a proteasome inhibitor.

In another aspect, there is provided an in vitro method for enhancing proteasome inhibitor suppression of a malignant B-cell, said method comprising contacting a malignant B-cell with (a) a proteasome inhibitor, in combination with (b) an ADC comprising an antibody or antigen-binding fragment thereof that binds to BCMA, conjugated to a nucleic acid cross-linking agent.

The term “suppresses” or “suppressing” in the context or any medicament, method, or use described herein embraces “inhibiting the growth of”, “inhibiting the proliferation of”, or “killing” a malignant B-cell. Reference to a “malignant B-cell” embraces a “tumour comprising a malignant B-cell”.

The term “inhibits” or “inhibiting” are synonymous with the term “retards the growth of” or “stops the proliferation of” a malignant B-cell, or “retards the growth of” a tumour comprising a malignant B-cell. In one embodiment, a therapeutic combination of the invention may “kill” a malignant B-cell or be “used to kill” a malignant B-cell, or tumour comprising a malignant B-cell. The term “suppressing” also encompasses preventing the growth (e.g. proliferation) of a malignant B-cell, or a tumour comprising a malignant B-cell.

In one embodiment, an enhanced suppression of a B-cell malignancy may comprise one or more selected from an enhanced delay in tumour growth, an enhanced reduction in tumour size, an enhanced reduction in tumour metastasis, an enhanced survival rate in a subject comprising a B-cell malignancy, or a combination thereof. In one embodiment, an enhanced suppression of a B-cell malignancy comprises one or more selected from an enhanced delay in tumour growth, an enhanced reduction in tumour size, or a combination thereof.

In one embodiment, a medicament and/or therapeutic combination of the invention suppresses a B-cell malignancy by at least 10%, or at least 20%, or at least 30%, or at least 40%, or at least 50%, or at least 60%, or at least 70%, or at least 80%, or at least 90% or about 100% greater that an otherwise identical medicament and/or composition lacking a proteasome inhibitor. In one embodiment, a medicament and/or therapeutic combination of the invention suppresses a B-cell malignancy by at least 10%, or at least 20%, or at least 30%, or at least 40%, or at least 50%, or at least 60%, or at least 70%, or at least 80%, or at least 90% or about 100% greater that an otherwise identical medicament and/or composition lacking an ADC of the invention.

Suppression may be measured by measuring cellular proliferation, which can be assayed using art recognized techniques which measure a rate of cell division, and/or the fraction of cells within a cell population undergoing cell division, and/or rate of cell loss from a cell population due to terminal differentiation or cell death (e.g., thymidine incorporation).

An assessment of said “enhanced suppression of a B-cell malignancy”, is demonstrated by reference to the accompanying Examples, and may be assessed using the methodology described in the Examples (e.g. Example 3). For example, Example 3 describes a method comprising Annexin V/PI-based FMC analysis, which measures the “Observed % Apoptotic Cells” value in an in vitro culture of malignant B-cells following contact with a test sample. This allows for direct comparison of B-cell malignancy suppression between a medicament of the invention, and an otherwise identical medicament lacking a proteasome inhibitor or ADC.

In one embodiment, suppression of a B-cell malignancy is considered to be enhanced when the “Observed % Apoptotic Cells” value obtained for a combination of the two principal active compounds (e.g. an ADC of the invention, and a proteasome inhibitor) is greater that the “Observed % Apoptotic Cells” value obtained when either an ADC of the invention, or a proteasome inhibitor (suitably a proteasome inhibitor) is absent (but under otherwise identical conditions).

Thus in one embodiment enhanced suppression of a B-cell malignancy may be determined by comparing the “Observed % Apoptotic Cells” value obtained for a combination of an ADC of the invention, and a proteasome inhibitor with the “Observed % Apoptotic Cells” value obtained for the same formulation (e.g. medicament) absent an ADC of the invention, or a proteasome inhibitor (suitably lacking a proteasome inhibitor) under the same conditions.

In one embodiment the present invention provides an enhanced suppression of a B-cell malignancy that is at least about 5%, 10%, 15%, 20%, 25%, 30%, 35%, 40%, 45%, 50%, 55%, 60%, 65%, 70%, 75%, 80%, 85%, 90% or 95% greater (Observed % Apoptotic Cells) than an Observed % Apoptotic Cells provided by the same formulation (e.g. medicament) absent an ADC of the invention, or a proteasome inhibitor (suitably lacking a proteasome inhibitor) under the same conditions. In one embodiment the present invention provides an enhanced suppression of a B-cell malignancy that is at least about 65% greater (Observed % Apoptotic Cells) than an Observed % Apoptotic Cells provided by the same formulation (e.g. medicament) absent an ADC of the invention, or a proteasome inhibitor (suitably lacking a proteasome inhibitor) under the same conditions.

In one embodiment a combination of an ADC of the invention, and a proteasome inhibitor, may exhibit synergistic suppression of a B-cell malignancy.

The term “synergistic” as used herein means that the suppression of a B-cell malignancy exhibited is greater than the sum of its parts. In other words, the suppression of a B-cell malignancy is more than additive.

Synergism may be measured by determining the “Combination Index” (CI) using analysis tools such a CompuSyn (ComboSyn, Inc.), in which a CI of <1 indicates synergism between a combination of the principal active components of the invention, a CI of >1 indicates antagonism, and a CI of 1 indicates that the effect is additive. Said CI may be measured by comparing the performance, in any cell viability assay known in the art (e.g. alternatively or additionally to the methodology of Examples 1-3, such as the “CellTiter-Glo-based cell viability” assay described in Example 3), of a composition comprising the medicament/therapeutic combination of the invention with the performance of an otherwise identical composition lacking an ADC of the invention, or a proteasome inhibitor (suitably lacking a proteasome inhibitor).

Referring to the Examples (e.g. Example 3), synergistic suppression of a B-cell malignancy may be considered present when the CI is less than about 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, or 0.05. In one embodiment, said CI may be less than about 0.7. In one embodiment, said CI may be less than about 0.6.

In one embodiment, a “cell viability assay” comprises: incubating a test sample comprising malignant B-cells in the presence of an amount of a composition comprising the therapeutic combination (an ADC of the invention, and a proteasome inhibitor); and comparing the number of non-viable cells (e.g. apoptotic cells) in the test sample subsequent to incubation (e.g. following at least 0.5, 1, 1.5 or 2 days of incubation) with the number of non-viable cells in a control sample incubated in the presence of an otherwise identical composition lacking (a) said proteasome inhibitor, or (b) said ADC (suitably lacking said proteasome inhibitor).

In one embodiment, the therapeutic combination is administered to a subject. The terms “subject”, “individual” and “patient” are used interchangeably herein to refer to a mammalian subject. In one embodiment the “subject” is a human, a companion animal (e.g. a pet such as a dog, cat, and/or rabbit), livestock (e.g. a pig, sheep, cattle, and/or a goat), and/or a horse. In one embodiment, the subject is a human.

In methods of the invention, the subject may not have been previously diagnosed as having a B-cell malignancy. Alternatively, the subject may have been previously diagnosed as having a B-cell malignancy. The subject may also be one who exhibits disease risk factors, or one who is asymptomatic for a B-cell malignancy. The subject may also be one who is suffering from or is at risk of developing a B-cell malignancy. In one embodiment, the subject has been previously administered a therapy for a B-cell malignancy.

In one embodiment, methods and uses of the invention comprise one or more administration step selected from oral, intravenous, intraarterial, intraperitoneal, intramuscular, subcutaneous, rectal, or vaginal, inhalation, topical, or a combination thereof. In one embodiment, the administration is one or more selected from intravenous, intraarterial (e.g. by injection or drip), subcutaneous, or a combination thereof.

### Antibody Preparation

The antibodies of the present invention can be obtained using conventional techniques known to persons skilled in the art and their utility confirmed by conventional binding studies—an exemplary method is described in Example 2. By way of example, a simple binding assay is to incubate the cell expressing an antigen with the antibody. If the antibody is tagged with a fluorophore, the binding of the antibody to the antigen can be detected by FACS analysis.

Methods for generating BCMA antibodies and antibody fragments thereof of the present invention are described in WO 2010/104949 and WO 2019/025983 (in particular, WO 2019/025983), both of which are incorporated herein by reference.

Antibodies of the present invention can be raised in various animals including mice, rats, rabbits, goats, sheep, monkeys or horses. Antibodies may be raised following immunisation with individual capsular polysaccharides, or with a plurality of capsular polysaccharides. Blood isolated from these animals contains polyclonal antibodies—multiple antibodies that bind to the same antigen. Antigens may also be injected into chickens for generation of polyclonal antibodies in egg yolk. To obtain a monoclonal antibody that is specific for a single epitope of an antigen, antibody-secreting lymphocytes are isolated from an animal and immortalized by fusing them with a cancer cell line. The fused cells are called hybridomas, and will continually grow and secrete antibody in culture. Single hybridoma cells are isolated by dilution cloning to generate cell clones that all produce the same antibody; these antibodies are called monoclonal antibodies. Methods for producing monoclonal antibodies are conventional techniques known to those skilled in the art (see e.g. Making and Using Antibodies: A Practical Handbook. GC Howard. CRC Books. 2006. ISBN 0849335280). Polyclonal and monoclonal antibodies are often purified using Protein A/G or antigen-affinity chromatography.

The antibody or antigen binding fragment thereof of the invention may be prepared as a monoclonal antibody, which can be prepared using hybridoma methods, such as those described by Kohler and Milstein, Nature 256:495 (1975). Using the hybridoma method, a mouse, hamster, or other appropriate host animal, is immunized as described above to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or an in vitro binding assay, e.g., radioimmunoassay (RIA) or enzyme-linked immunosorbent assay (ELISA), can then be propagated either in in vitro culture using standard methods (Goding, Monoclonal Antibodies: Principles and Practice, Academic Press, 1986) or in vivo as ascites tumours in an animal. The monoclonal antibodies can then be purified from the culture medium or ascites fluid using known methods.

Alternatively, the antibody or antigen binding fragment thereof (e.g. as monoclonal antibodies) can also be made using recombinant DNA methods as described in U.S. Pat. No. 4,816,567. The polynucleotides encoding a monoclonal antibody are isolated from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or antigen-binding fragments thereof of the desired species can be isolated from phage display libraries expressing CDRs of the desired species as described in McCafferty et al., Nature 348:552-554 (1990); Clackson et al., Nature, 352:624-628 (1991); and Marks et al., J. Mol. Biol. 222:581-597 (1991).

The polynucleotide(s) encoding an antibody or an antigen-binding fragment thereof of the invention can further be modified in a number of different manners using recombinant DNA technology to generate alternative antibodies. In some embodiments, the constant domains of the light and heavy chains of, for example, a mouse monoclonal antibody can be substituted (1) for those regions of, for example, a human antibody to generate a chimeric antibody or (2) for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

In one embodiment, the antibody or antigen-binding fragment thereof is a human antibody or antigen-binding fragment thereof. Human antibodies can be directly prepared using various techniques known in the art. Immortalized human B lymphocytes immunized in vitro or isolated from an immunized individual that produce an antibody directed against a target antigen can be generated. See, e.g., Cole et al., Monoclonal Antibodies and Cancer Therapy, Alan R. Liss, p. 77 (1985); Boemer et al., J. Immunol. 147 (1):86-95 (1991); U.S. Patent 5,750,373.

In one embodiment, the antibody or antigen-binding fragment thereof can be selected from a phage library, where that phage library expresses human antibodies, as described, for example, in Vaughan et al., Nat. Biotech. 14:309-314 (1996); Sheets et al., Proc. Natl. Acad. Sci. USA, 95:6157-6162 (1998); Hoogenboom and Winter, J. Mol. Biol. 227:381 (1991); and Marks et al., J. Mol. Biol. 222:581 (1991). Techniques for the generation and use of antibody phage libraries are also described in U.S. Pat. Nos. 5,969,108, 6,172,197, 5,885,793, 6,521,404; 6,544,731; 6,555,313; 6,582,915; 6,593,081; 6,300,064; 6,653,068; 6,706,484; and 7,264,963; and Rothe et al., J. Molec. Biol. 376:1182-1200 (2008), each of which is incorporated by reference in its entirety.

Affinity maturation strategies and chain shuffling strategies are known in the art and can be employed to generate high affinity human antibodies or antigen-binding fragments thereof. See Marks et al., BioTechnology 10:779-783 (1992), incorporated by reference in its entirety.

In one embodiment, the antibody or antigen binding fragment thereof (e.g. an monoclonal antibody) can be a humanized antibody. Methods for engineering, humanizing or resurfacing non-human or human antibodies can also be used and are well known in the art. A humanized, resurfaced or similarly engineered antibody can have one or more amino acid residues from a source that is non-human, e.g., but not limited to, mouse, rat, rabbit, non-human primate, or other mammal. These non-human amino acid residues are replaced by residues that are often referred to as “import” residues, which are typically taken from an “import” variable, constant or other domain of a known human sequence. Such imported sequences can be used to reduce immunogenicity or reduce, enhance or modify binding, affinity, on-rate, off-rate, avidity, specificity, half-life, or any other suitable characteristic, as known in the art. Suitably, the CDR residues may be directly and most substantially involved in influencing antigen (e.g. BCMA) binding. Accordingly, part or all of the non-human or human CDR sequences may be maintained while the non-human sequences of the variable and constant regions can be replaced with human or other amino acids.

Antibodies can also optionally be humanized, resurfaced, engineered or human antibodies engineered with retention of high affinity for the antigen (e.g. BCMA) and other favourable biological properties. To achieve this goal, humanized (or human) or engineered antibodies and resurfaced antibodies can be optionally prepared by a process of analysis of the parental sequences and various conceptual humanized and engineered products using three-dimensional models of the parental, engineered, and humanized sequences. Three-dimensional immunoglobulin models are commonly available and are familiar to those skilled in the art. Computer programs are available which illustrate and display probable three-dimensional conformational structures of selected candidate immunoglobulin sequences.

Inspection of these displays permits analysis of the likely role of the residues in the functioning of the candidate immunoglobulin sequence, i.e., the analysis of residues that influence the ability of the candidate immunoglobulin to bind its antigen (e.g. BCMA). In this way, FW residues can be selected and combined from the consensus and import sequences so that the desired antibody characteristic, such as increased affinity for the target antigen(s), is achieved.

Humanization, resurfacing or engineering of an antibody or antigen-binding fragment thereof of the present invention can be performed using any known method, such as but not limited to those described in, Jones et al., Nature 321:522 (1986); Riechmann et al., Nature 332:323 (1988); Verhoeyen et al., Science 239:1534 (1988); Sims et al., J. Immunol. 151: 2296 (1993); Chothia and Lesk, J. Mol. Biol. 196:901 (1987); Carter et al., Proc. Natl. Acad. Sci. USA 89:4285 (1992); Presta et al., J. Immunol. 151:2623 (1993); U.S. Pat. Nos. 5,639,641, 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; 4,816,567, 7,557,189; 7,538,195; and 7,342,110; International Application Nos. PCT/US98/16280; PCT/US96/18978; PCT/US91/09630; PCT/US91/05939; PCT/US94/01234; PCT/GB89/01334; PCT/GB91/01134; PCT/GB92/01755; International Patent Application Publication Nos. WO90/14443; WO90/14424; WO90/14430; and European Patent Publication No. EP 229246; each of which is entirely incorporated herein by reference, including the references cited therein.

An antibody or antigen-binding fragment thereof can also be made in transgenic mice containing human immunoglobulin loci that are capable upon immunization of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

In one embodiment, a fragment (e.g. antibody fragment) of the antibody of the invention is provided. Various techniques are known for the production of antibody fragments. The terms “antibody fragment,” “antigen-binding fragment,” “functional fragment of an antibody,” and “antigen-binding portion” are used interchangeably herein and refer to one or more fragments or portions of an antibody that retain the ability to specifically bind to an antigen (e.g. BCMA) which the antibody (e.g. the “whole” or “parent” antibody) binds to. Therefore, reference to an “antigen binding fragment thereof” means an antigen binding fragment that binds BCMA (e.g. the BCMA-antigen binding fragment of the antibody).

Traditionally, these fragments are derived via proteolytic digestion of intact antibodies, as described, for example, by Morimoto et al., J. Biochem. Biophys. Meth. 24:107-117 (1993) and Brennan et al., Science 229:81 (1985). In one embodiment, anti-BCMA antibody fragments are produced recombinantly. Fab, Fv, and scFv antibody fragments can all be expressed in and secreted from E. coli or other host cells, thus allowing the production of large amounts of these fragments. Such anti-BCMA antibody fragments can also be isolated from the antibody phage libraries discussed above. The anti-BCMA antibody fragments can also be linear antibodies as described in U.S. Pat. No. 5,641,870. Other techniques for the production of antibody fragments will be apparent to the skilled practitioner.

A modified antibody or antigen-binding fragment thereof as provided herein can comprise any type of variable region that provides for the association of the antibody or polypeptide with BCMA. In this regard, the variable region can comprise or be derived from any type of mammal that can be induced to mount a humoral response and generate immunoglobulins against the desired antigen. As such, the variable region of an anti-BCMA antibody or antigen-binding fragment thereof can be, for example, of human, murine, non-human primate (e.g., cynomolgus monkeys, macaques, etc.) or lupine origin. In one embodiment, both the variable and constant regions of the modified antibody or antigen-binding fragment thereof are human. In one embodiment, the variable regions of a compatible antibody (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In one embodiment, the variable domains in both the heavy and light chains of an antibody or antigen-binding fragment thereof are altered by at least partial replacement of one or more CDRs and/or by partial framework region replacement and sequence changing. Although the CDRs can be derived from an antibody of the same class or even subclass as the antibody from which the framework regions are derived, it is envisaged that the CDRs will be derived from an antibody of different class and in certain embodiments from an antibody from a different species. It is not necessary to replace all of the CDRs with the complete CDRs from the donor variable region to transfer the antigen-binding capacity of one variable domain to another. Rather, it is only necessary to transfer those residues that are necessary to maintain the activity of the antigen-binding site. Given the explanations set forth in U.S. Pat. Nos. 5,585,089, 5,693,761 and 5,693,762, it will be well within the competence of those skilled in the art to carry out routine experimentation to obtain a functional antibody with reduced immunogenicity.

Alterations to the variable region notwithstanding, those skilled in the art will appreciate that a modified antibody or antigen-binding fragment thereof of this invention will comprise an antibody (e.g., full-length antibody or antigen-binding fragment thereof) in which at least a fraction of one or more of the constant region domains has been deleted or otherwise altered so as to provide desired biochemical characteristics such as increased tumour localization or reduced serum half-life when compared with an antibody of approximately the same immunogenicity comprising a native or unaltered constant region. In one embodiment, the constant region of the modified antibody will comprise a human constant region. Modifications to the constant region compatible with this invention comprise additions, deletions or substitutions of one or more amino acids in one or more domains. That is, the modified antibody disclosed herein can comprise alterations or modifications to one or more of the three heavy chain constant domains (CH1, CH2 or CH3) and/or to the light chain constant domain (CL). In one embodiment, a modified constant region wherein one or more domains are partially or entirely deleted are contemplated. In one embodiment, a modified antibody will comprise domain deleted constructs or variants wherein the entire CH2 domain has been removed (ΔCH2 constructs). In one embodiment, the omitted constant region domain can be replaced by a short amino acid spacer (e.g., 10 residues) that provides some of the molecular flexibility typically imparted by the absent constant region.

Besides the deletion of whole constant region domains, an antibody or antigen-binding fragment thereof provided herein can be modified by the partial deletion or substitution of a few or even a single amino acid in a constant region. For example, the mutation of a single amino acid in selected areas of the CH2 domain can be enough to substantially reduce Fc binding and thereby increase tumour localization. Similarly one or more constant region domains that control the effector function (e.g., complement C1Q binding) can be fully or partially deleted. Such partial deletions of the constant regions can improve selected characteristics of the antibody or antigen-binding fragment thereof (e.g., serum half-life) while leaving other desirable functions associated with the subject constant region domain intact. Moreover, the constant regions of the antibody and antigen-binding fragment thereof can be modified through the mutation or substitution of one or more amino acids that enhances the profile of the resulting construct. In this respect it is possible to disrupt the activity provided by a conserved binding site (e.g., Fc binding) while substantially maintaining the configuration and immunogenic profile of the modified antibody or antigen-binding fragment thereof. In one embodiment, there may be an addition of one or more amino acids to the constant region to enhance desirable characteristics such as decreasing or increasing effector function or provide for more cytotoxin or carbohydrate attachment. In one embodiment, it can be desirable to insert or replicate specific sequences derived from selected constant region domains.

The present invention further embraces variants and equivalents that are substantially homologous to an antibody or antigen binding fragment of the invention (e.g. murine, chimeric, humanized or human antibody, or antigen-binding fragments thereof). These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art.

In one embodiment, the antibody or antigen-binding fragment thereof can be further modified to contain additional chemical moieties not normally part of the protein. Those derivatized moieties can improve the solubility, the biological half-life or absorption of the protein. The moieties can also reduce or eliminate any desirable side effects of the proteins and the like. An overview for those moieties can be found in Remington's Pharmaceutical Sciences, 22nd ed., Ed. Lloyd V. Allen, Jr. (2012).

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive

Multiple Sequence Alignment Through Sequence Weighting, Position- Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501 -509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic: arginine
lysine
histidine
   Acidic: glutamic acid
aspartic acid
   Polar: glutamine
asparagine
   Hydrophobic: leucine
isoleucine
valine
   Aromatic: phenylalanine
tryptophan
tyrosine
   Small: glycine
alanine
serine
threonine
methionine

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, N.Y. (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a cytotoxin” includes a plurality of such cytotoxins and reference to “the cytotoxin” includes reference to one or more cytotoxins and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### EXAMPLES

The invention will now be described, by way of example only, with reference to the following Examples.

### Materials and Methods

### Generation of Anti-BCMA Antibodies

Antibodies were generated as described in WO 2010/104949 and WO 2019/025983, both of which are incorporated herein by reference. Suitable antibodies were generated as described in Kinneer et al (2018), Leukemia 33, 766-771 and WO 2019/025983.

### Generation of Anti-BCMA ADCs

Anti-BCMA ADC (anti-BCMA antibody conjugated to PBD is herein referred to as “M2”) was prepared through site-specific conjugation of the PBD dimer, tesirine (SG3249), to the BCMA-Ab1 antibody described in Kinneer et al (2018), Leukemia 33, 766-771 (“Kinneer et al (2018)”), using a protease-cleavable linker, as previously described (see e.g. Kinneer et al (2018);

incorporated herein by reference). An example BCMA antibody is BCMA-Ab2, also described in Kinneer et al (2018). Said antibodies are further described in WO 2019/025983 (incorporated herein by reference) as 15B2GL, and J6M0-mc, respectively. The ADC “M3” was similarly generated by attaching monomethyl auristatin F (MMAF) payload to the antibody BCMA-Ab1. Both payloads were site-specifically conjugated to an engineered cysteine inserted after position 239 (C239i) in the CH2 domain of the BCMA antibody, as previously described. Briefly, the BCMA-Ab1 was reduced with 40 molar excess of TCEP for three hours at 37° C. followed by three successive dialysis to remove the TCEP. The antibody was then oxidized with 20 molar excess of DHAA for four hours at room temperature and conjugated using eight molar equivalence of payload. After conjugation, the free payload and protein aggregate were removed by Ceramic Hydroxyapatite purification.

### Murine Xenograft Model of Human MM

All animal experiments were approved by and conformed to the relevant regulatory standards of the Institutional Animal Care and Use Committee at the Dana-Farber Cancer Institute. CB-17 SCID-mice were subcutaneously inoculated with 5.0×106 MM.1S cells in 100 μl of serum-free RPMI 1640 medium. When tumours were measurable approximately 3 weeks after MM-cell injection, mice (8 mice/group) were randomized and treated with vehicle alone, M2, btz, or M2 with btz. Tumour size was measured every third day in 2 dimensions using calipers, and tumour volume was calculated using the following formula: V=0.5a×b2, where “a” and “b” are the long and short diameter of the tumour, respectively. Animals were sacrificed when their tumours reached 2 cm³.

### Analysis of Tumours Harvested from Mice Using Immunoblotting and Immunostaining

Following 3d-treatment, tumour from each group was harvested and cell lysates were made for immunoblotting. Sections of tumours collected from mice were subjected to immunohistochemical staining for proliferation by Ki67 (BCR CRM325). Immunohistochemical images were taken on Zeiss Inverted Fluorescence Microscope for Ki67. A Plan-Apochromat 63X/1.40 Oil DIC M27 objective lens was used.

### Cells and Cell Culture

MM cell lines were cultured in RPMI containing 10% fetal bovine serum (GIBCO, 10437028), 2 mM/L L-glutamine, 100 U/mL penicillin, and 100 mg/mL streptomycin (GIBCO, 15140122). They are routinely checked by Human STR Profiling Cell Authentication for their authenticity and for mycoplasma contamination. Patient MM and normal donor samples were obtained after informed consent was provided, in accordance with the Declaration of Helsinki and under the auspices of a Dana-Farber Cancer Institute Institutional Review Board approved protocol. Primary CD138+ plasma cells (>95% purity) were purified from bone marrow mononuclear cells (BMMCs) from BM aspirates of MM patients using anti-CD138 microbeads (Miltenyi Biotech, Auburn, Calif.). Residual CD138-negative BMMCs were further cultured to derive

BMSCs. Peripheral blood mononuclear cells (PBMCs) were isolated from PB samples using Ficoll-Hypaque density gradient. Bortezomib was purchased from Selleckchem (Selleck Chemicals).

### Cell Viability and Apoptosis Assay

Cell viability was analyzed by CCK8 (Abcam, Cambridge, Mass.), CellTiter-Glo (CTG) (Promega), and BLI measurement. Apoptosis was evaluated by flow cytometric analysis following FITC Annexin-V (BD Biosciences), PE-Annexin-V (BioLegend), and/or LIVE/DEAD™ Fixable Aqua (Invitrogen, L34957) staining, according to manufacturer's instructions. MM cells were labeled with CFSE (Invitrogen), and then cultured for 2 days, alone or with BMSC, followed by AnnexinV/Aqua staining and flow cytometry analysis.

### Luciferase Proliferation Assay

BMSC were seeded in 96-well plates and incubated for 24 hours to allow cells to adhere. MM1 Sluc cells were cultured at a ratio of 100:1 on confluent layers of BMSC in RPMI medium for 4d. Proliferation was measured using the Luciferase assay, according to manufacturer's protocol (Promega, Madison, Wis.).

### Statistical Analysis

Each experiment was performed at least three times, and data are represented as mean ±SD. Data were analyzed using Student t tests for 2 group comparisons or a 1-way analysis of variance (ANOVA) for multiple comparisons using the Graphpad software (GraphPad Software, La Jolla, Calif., USA). P-value <0.05 was statistically significant. Drug interactions were assessed by CompuSyn software to determine combination index (CI). CI<1 indicates synergism whereas CI>1 antagonism and CI=1 additive effect.

### Example 1

The results of the experiments reflected in this example demonstrate that an anti-BCMA antibody-PBD conjugate (M2) induces more potent cytotoxicity against drug-resistant MM cells than its MMAF ADC homolog (M3).

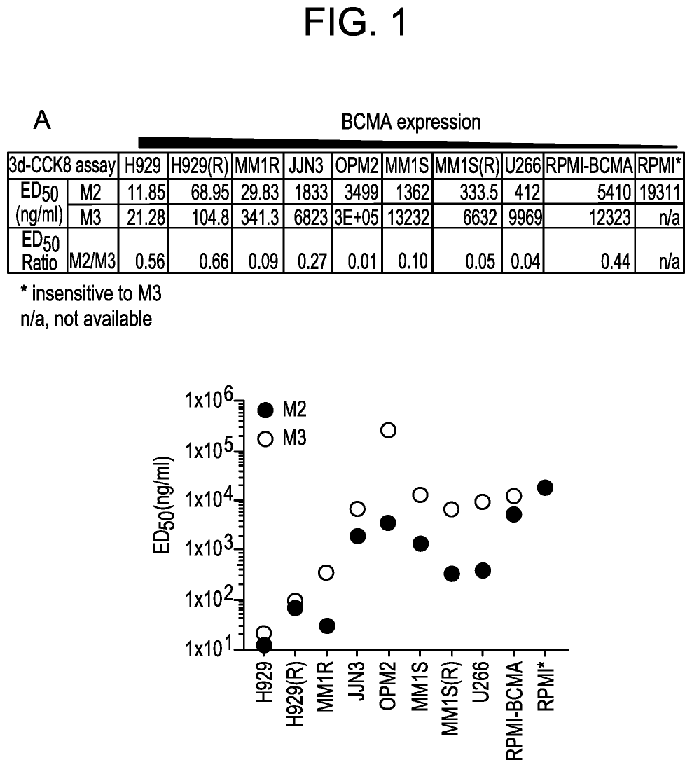

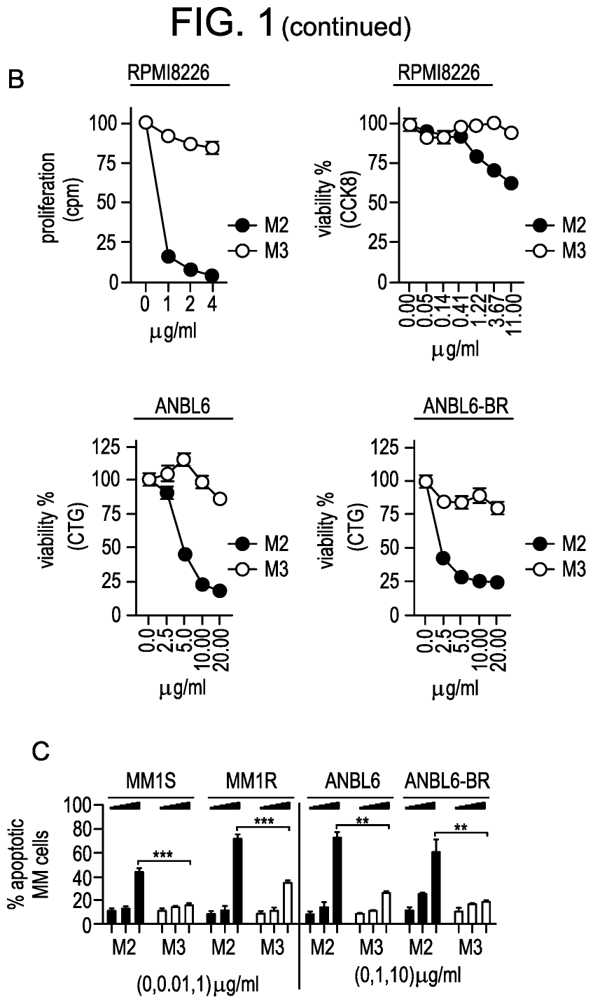

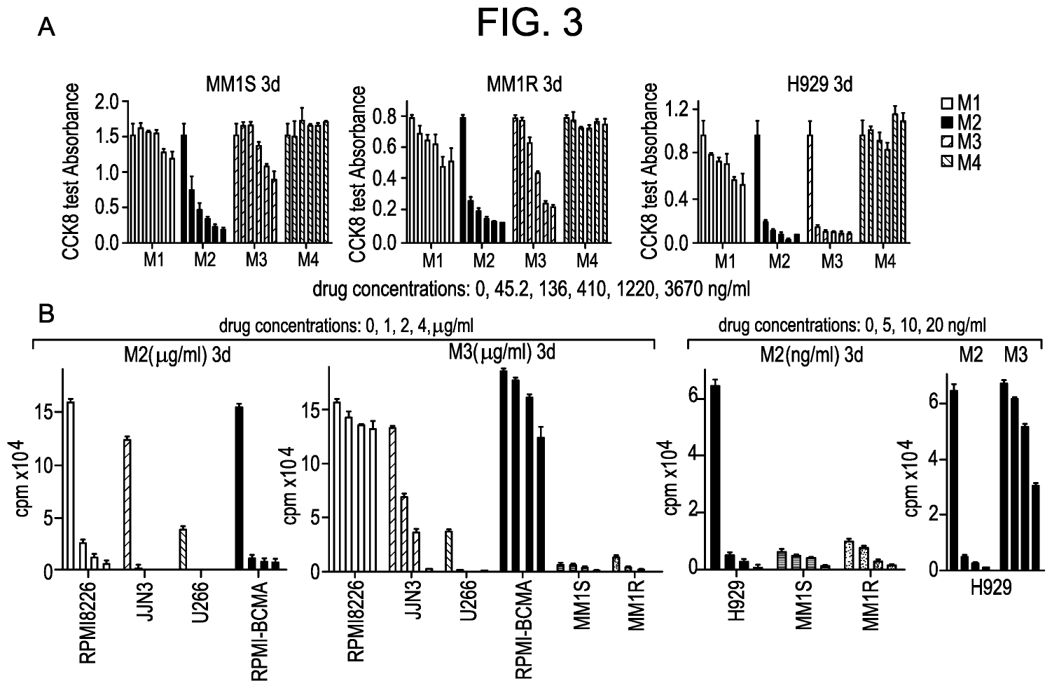

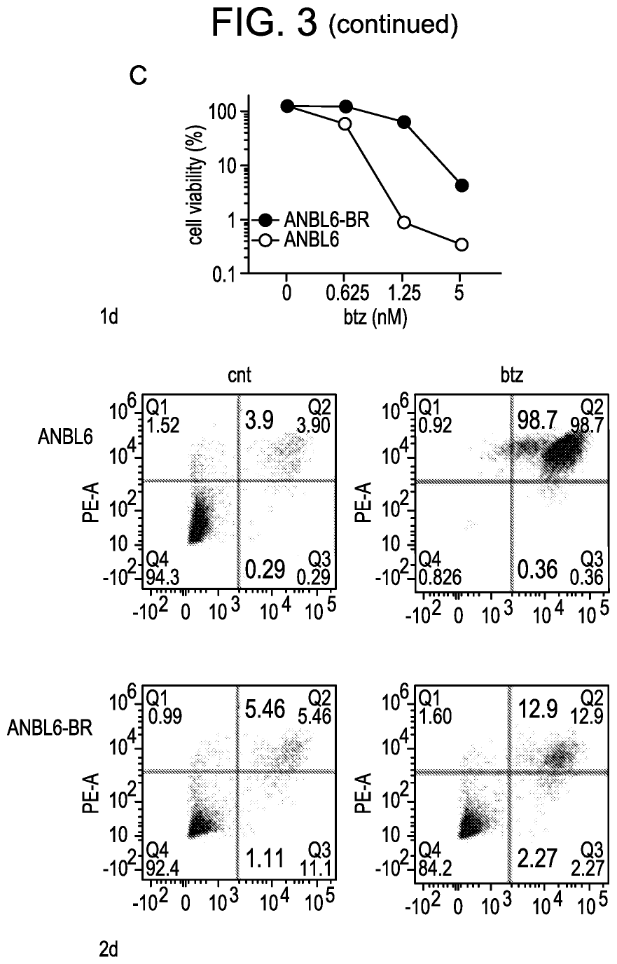

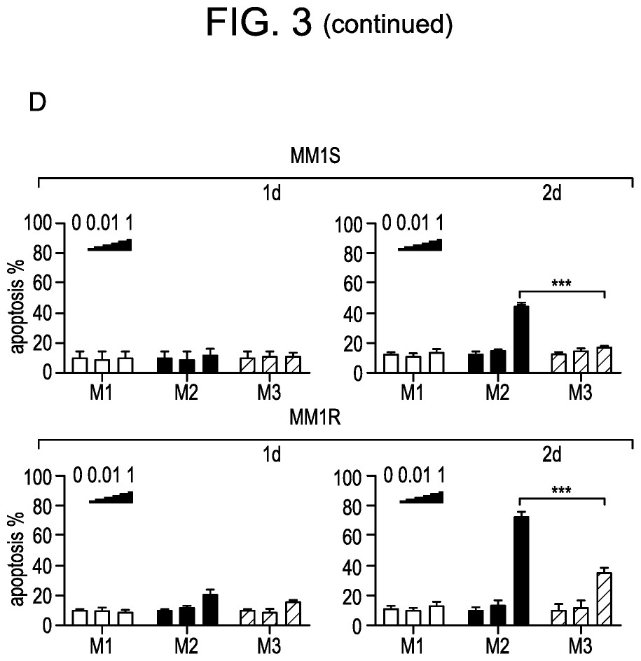

The cytotoxicity of an ADC comprised of an anti-BCMA antibody conjugated to a PBD (M2) was compared to the cytotoxicity of its MMAF ADC homolog (M3) against a panel of MM cell lines with various levels of BCMA expression and response to current anti-MM drugs. Both ADCs are composed of the same anti-BCMA mAb (BCMA-Ab1/15B2GL, as described above) but are conjugated to different payloads: a DNA cross-linking PBD for M2 (e.g. tesirine), and a microtubule-binding MMAF for M3. Using the 3d CCK8-based viability assay, ED₅₀ values of M2 are lower than those of M3 in all tested MM cell lines (n=10), regardless of sensitivity to anti-MM therapies including dexamethasone and IMiDs (FIG. 1A, FIG. 3A). In 8 MM cell lines, not including RPM18226 (RPMI) and its derived BCMA-overexpressing RPMI-BCMA, ED₅₀ values range from 11.85 to 3499 ng/ml and 21.28 to 271431 ng/ml for M2 and M3, respectively. All MM cells harbor various p53 mutations, except MM1S and H929 cells from which two IMiDs-resistant MM1S(R) and H929(R) cells are derived, respectively. M2, but not M3, is cytotoxic to RPM18226 cells expressing the lowest BCMA levels and resistance to IMiDs (FIG. 1A-B). Using DNA synthesis assay, M2 shows greater (>1-2-log) potency than M3 in blocking proliferation of all MM cells (FIG. 1B, FIG. 3B). For example, ED₅₀s for M2 vs M3 are 189.7 vs 21427 ng/ml in RPM18226 cells. Further, M2, but not M3, decreased viability of both ANBL6 and its derived bortezomib (btz)-resistant ANBL6-BR cells (FIG. 3C) cultured with IL-6. These paired IL-6-dependent ANBL6 cells are insensitive to M3 and express comparable cell membrane BCMA protein as RPM18226 cells (data not shown). Thus, MM cells with relatively lower BCMA expression are also significantly more susceptible to M2 vs M3.

Using flow cytometry (FCM) analysis following staining with Annexin V and live/dead Aqua, M2 was shown to induce earlier and increased apoptosis in paired MM cell lines sensitive or resistant to dexamethasone (dex) or bortezomib (btz) in a dose- and time-dependent manner, when compared with M3 (FIG. 1C, FIG. 3D). These in vitro results indicate that M2 overcomes resistance to current anti-MM drugs (dexamethasone, lenalidomide, pomalidomide, bortezomib) to a greater extent than M3 in MM cells, regardless of BCMA levels and p53 status.

### Example 2

The results of the experiments reflected in this example demonstrate that M2 is more effective than M3 in inducing cytotoxicity against MM cells in the bone marrow microenvironment and patient MM cells.

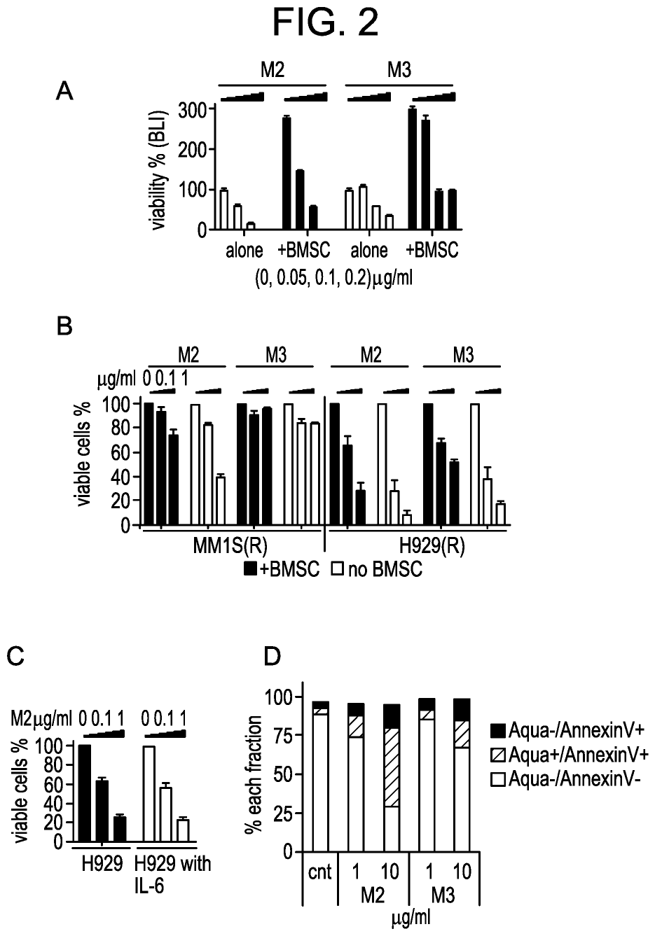

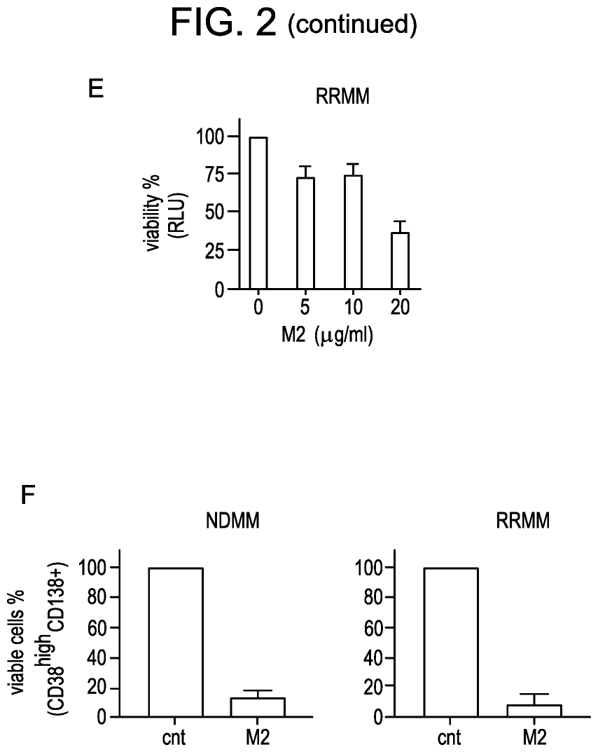

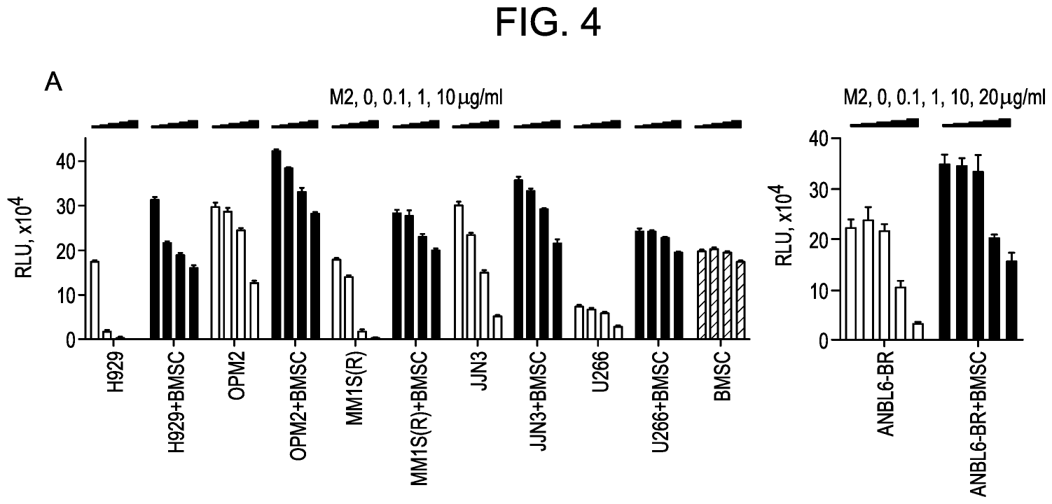

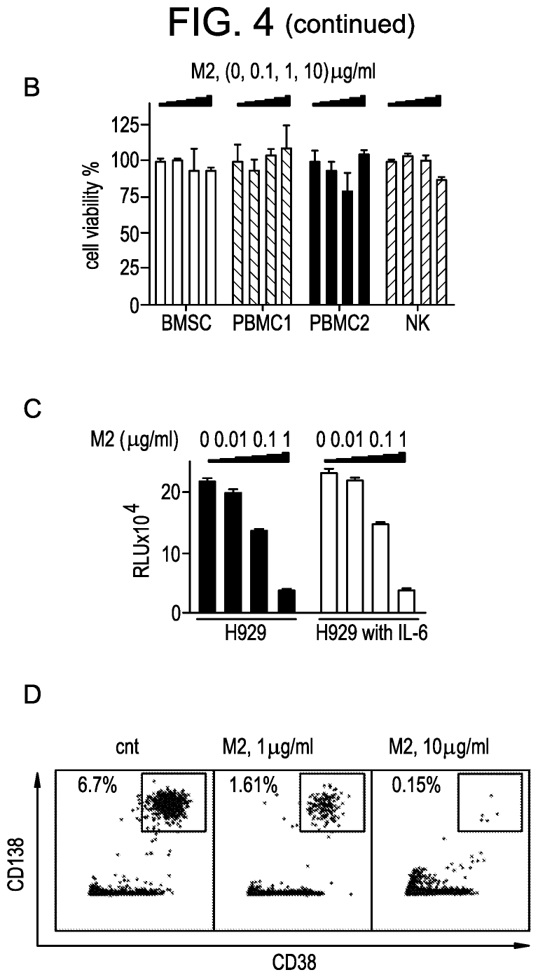

Next, the effects of M2 and M3 on MM cells co-cultured with bone marrow stromal cells (BMSCs) and IL-6, which promote MM cell growth, survival, and drug resistance were evaluated. Using BLI measurement, BMSCs were shown to significantly increase growth and survival of MM1Sluc cells (FIG. 4A). In BLI- and CTG-based assays, M2, more potently than M3, inhibits viability of MM1Sluc and all other tested MM cell lines (n=6) co-cultured with BMSCs (FIG. 2A, FIG. 4A), with minimal impact on BCMA-negative non-MM cell subsets including BMSCs, PBMCs, and NK cells (FIG. 4B). Using FCM analysis to identify viable MM cells, M2, more effectively than M3, decreases survival of IMiD-resistant MM1S(R) and H929(R) cells, even in the presence of BMSCs (FIG. 2B). In quantitative FCM—(FIG. 2C) and CTG-based analyses (FIG. 4C), M2 reduced growth and survival of H929 MM cells in the presence or absence of IL-6.

Following 3d treatment, live and dead BM CD138+ cell fractions from patients with RRMM were quantitated by FCM analysis. Importantly, M2 in a dose-dependent manner increased (>2-fold) apoptotic CD138+ patient MM cells compared with M3 (FIG. 4D). In CTG-based assays, M2 also showed dose-dependent toxicity in CD138-purified BM cells from 3 additional patients with RRMM (FIG. 2E), as well as significantly depletes viable CD38highCD138+ BM cells from 4 patients with newly diagnosed MM (NDMM) (FIG. 2F, left, FIG. 4D) and 2 patients with RRMM (FIG. 2F, right). These data indicate that M2 depletes patient MM cells regardless of disease status and is significantly more cytotoxic to MM cells in the BM microenvironment than M3.

### Example 3

The results of the experiments reflected in this example demonstrate that M2, combined with bortezomib, induces synergistic cytotoxicity against MM cells in vitro and in vivo.

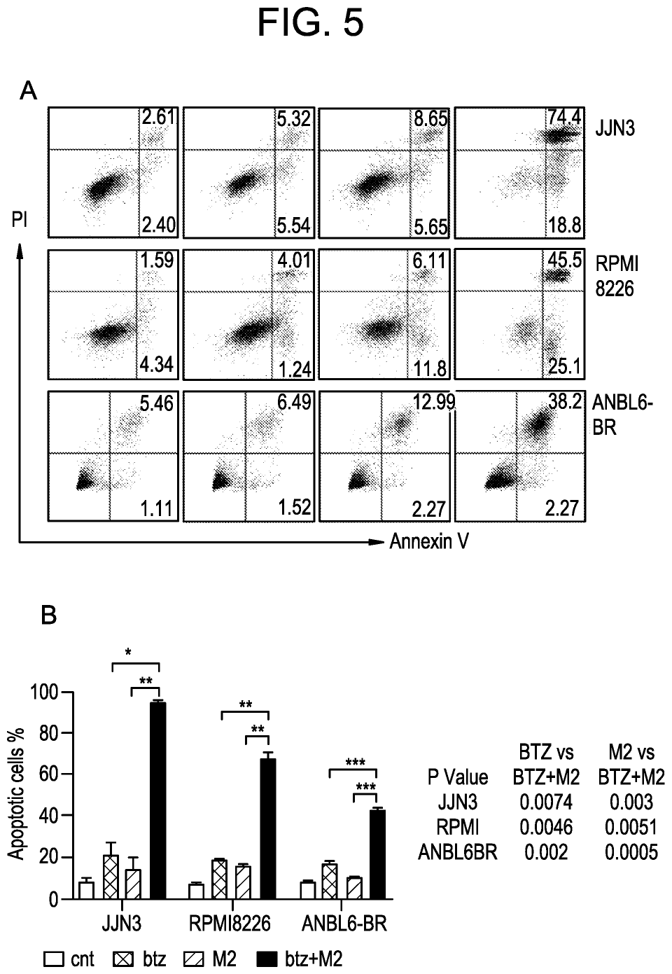

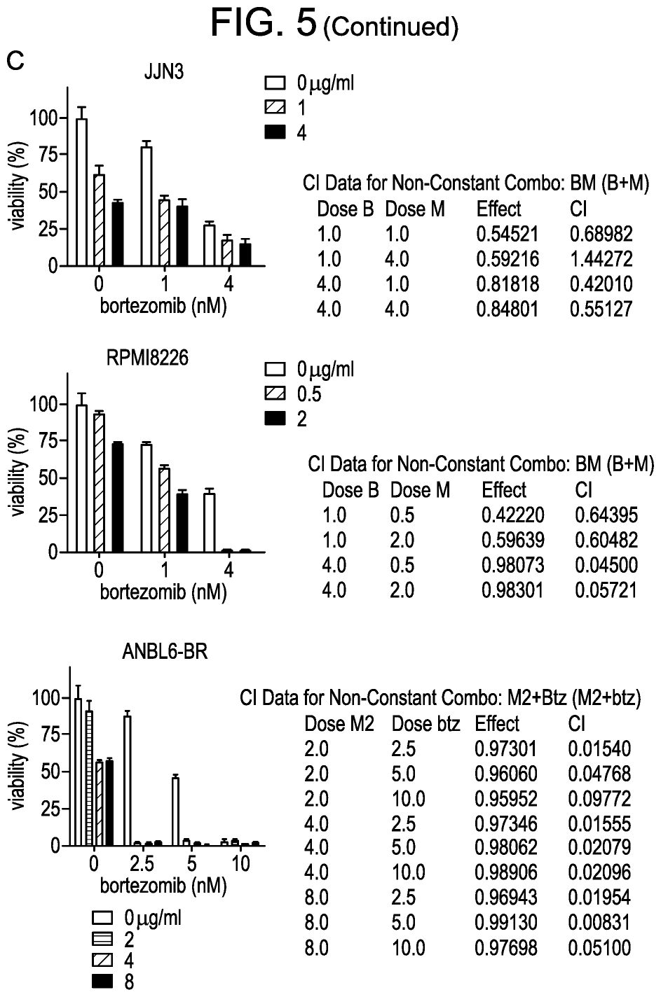

Bortezomib (btz) was chosen as a candidate co-therapy for M2, as btz is an existing myeloma therapy. Annexin V/Pl-based FMC analysis, combined M2 and btz at low doses for 2d further enhances apoptosis in JJN3 and RPM18226 cells, when compared with either agent alone (FIG. 5A-B, p<0.01). Significantly increased cell death following combined treatments is also seen in btz-resistant ANBL6-BR cells cultured in IL-6 (supporting the observation that addition of btz provides an effect that is more than additive). Next, results from CTG-based viability assays were analyzed to calculate combination indices (CIs). CIs<1 were derived in more than 6 representative MM cells, indicating synergistic effects of M2 plus btz (FIG. 5C, FIG. 6).

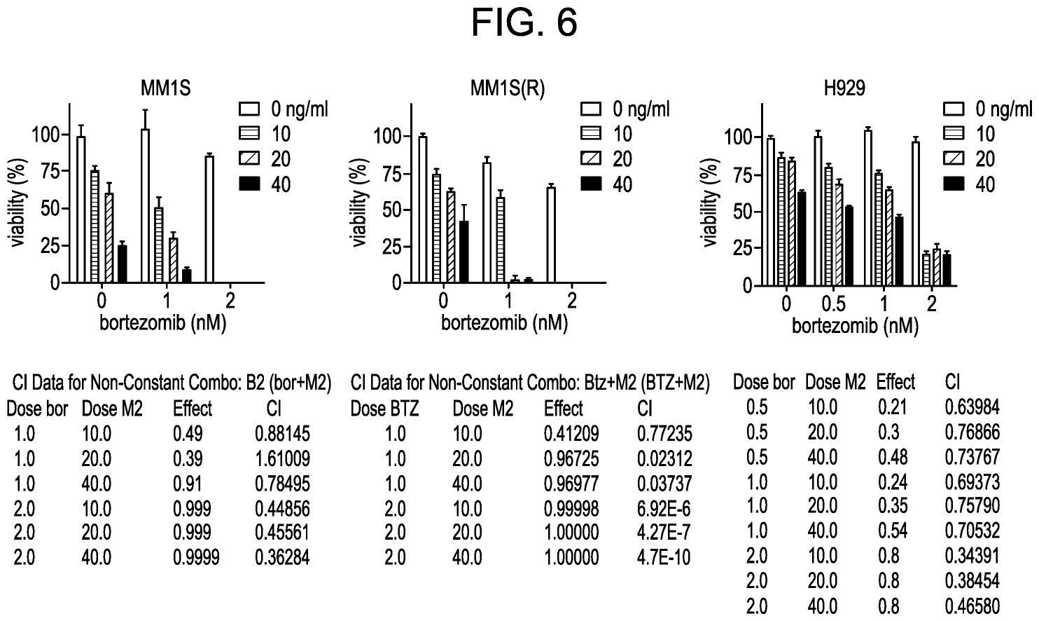

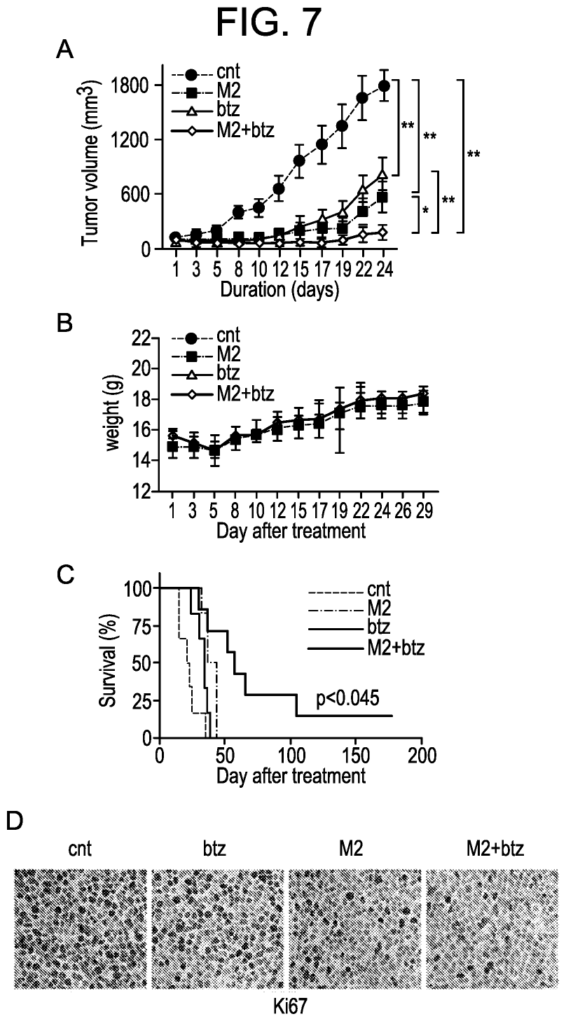

In vivo efficacy of sub-optimal doses M2 with btz was next evaluated in the MM1S xenograft mouse model. Mice with palpable MM1S tumours were randomized into 4 groups receiving either vehicle control, a single treatment of M2, or 6 treatments of btz (0.4 mg/kg) alone or with M2. At 24d after treatment, a single dose of M2 or a total of 6 doses of btz significantly delays MM1 S tumour growth in mice, compared with vehicle control (FIG. 7A, p<0.005). Combination treatment significantly decreased tumour volume vs either single agent alone (p<0.04).

Treatment with M2 plus btz is well tolerated, since the body weight of all animals was unaffected (FIG. 7B). Follow-up for 177 d shows a significant prolongation in median overall survival in the combination treated-group vs cohorts treated with either agent alone (cnt, 22 d; M2, 40.5 d; btz, 35 d; M2+btz, 57 d) (p<0.045) (FIG. 7C). At 177d, 15% mice are still alive without any tumour growth in the combination treated group.

Immunohistochemistry (IHC) for Ki67 (a cellular marker of proliferation) further confirm more potent inhibition of proliferation after combined vs single-agent treatment (FIG. 7D)—note reduced number of stained cells (dark colour) in the M2+btz treatment.

Combined treatments with M2 and btz significantly decreased in vivo growth of MM1 S xenografts (FIG. 8), demonstrating the in vivo synergism of M2 and btz in treating myeloma.

As a conclusion, the synergistic activity of M2 with btz observed in vitro at the cellular level is translated into superior in vivo efficacy in the plasmacytoma model of MM.

### Discussion of Examples 1-3

Disease recurrence due to drug resistance remains a major obstacle to more prolonged survival in MM. Therefore, novel therapies are needed to overcome drug resistance and address the unmet medical need in RRMM. Here, the inventors first show that an ADC (M2; an anti-BCMA antibody conjugated to a PBD) has superior cytotoxicity against all MM cell lines tested and patient MM cells than its MMAF ADC homolog. PBD dimers cause cell death in both rapidly dividing and more quiescent cells, unlike MMAF which predominantly targets proliferative tumour cells via binding to tubulin. M2 elicits a more potent effect on MM cell proliferation than its MMAF ADC homolog, including cells with low levels of BCMA expression and resistance to current therapies, even in the presence of BMSCs and IL-6. These data suggest that M2 may be more effective than its MMAF ADC homolog in treating aggressive MM.

Importantly, a combined treatment of M2 and btz in vitro induces synergistic death in all MM cells tested, evidenced by CI<1. Notably, M2 synergizes with btz even in btz-resistant ANBL6-BR cells, indicating other undefined molecules also responsible for enhanced cytotoxicity.

In mice bearing MM1S tumours, M2 is significantly more effective than btz as single agent therapy. Importantly, inhibition of in vivo tumour growth is further enhanced when M2 is combined with btz. Significant tumour necrosis is observed earlier in mice receiving both drugs than either agent alone, and at 177d, 15% mice in the combination treatment group remain alive and without tumour. Importantly, no weight loss is noted in all groups, indicating a favorable safety profile of M2 in vivo, suggesting that combination treatment of M2 and btz could be safely administered in vivo.

In summary, M2 specifically triggers potent growth inhibition and death, even in MM cells resistant to current MM therapies and protected by the BM microenvironment. In vivo, M2 is more effective than btz, and combining M2 with btz further enhances efficacy and prolongs host survival.

### Example 4

The results of the experiments reflected in this example demonstrate that M2 significantly activates DNA damage response and repair signaling cascades, followed by apoptosis, in drug-sensitive and drug-resistant MM cells.

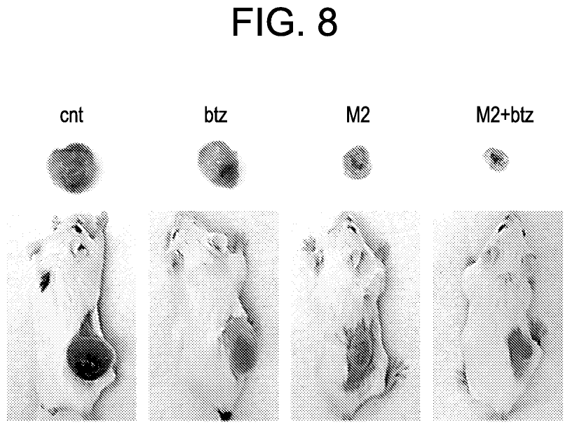

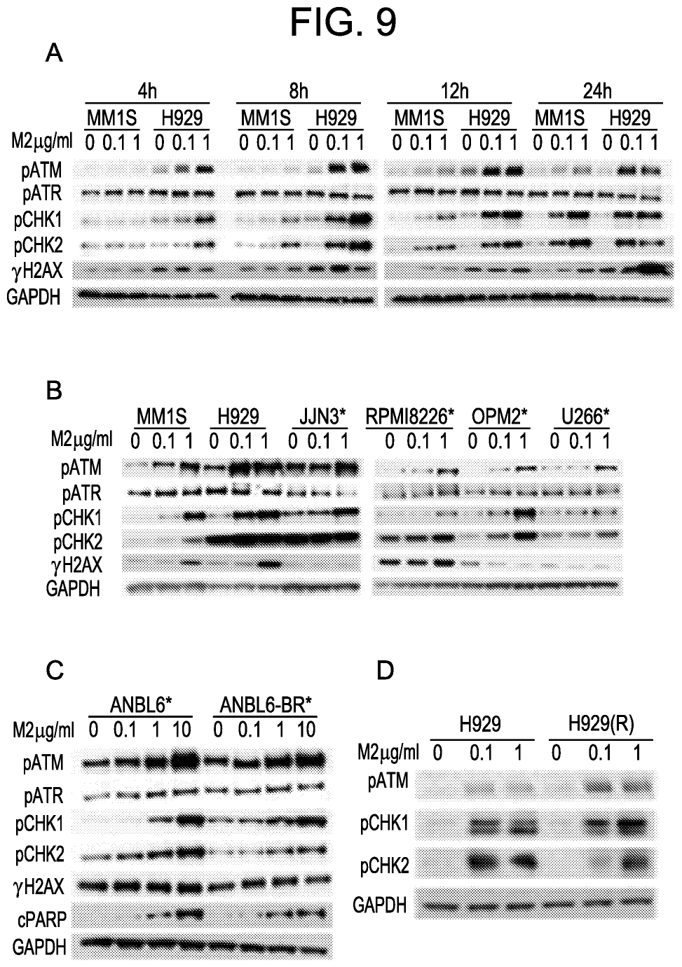

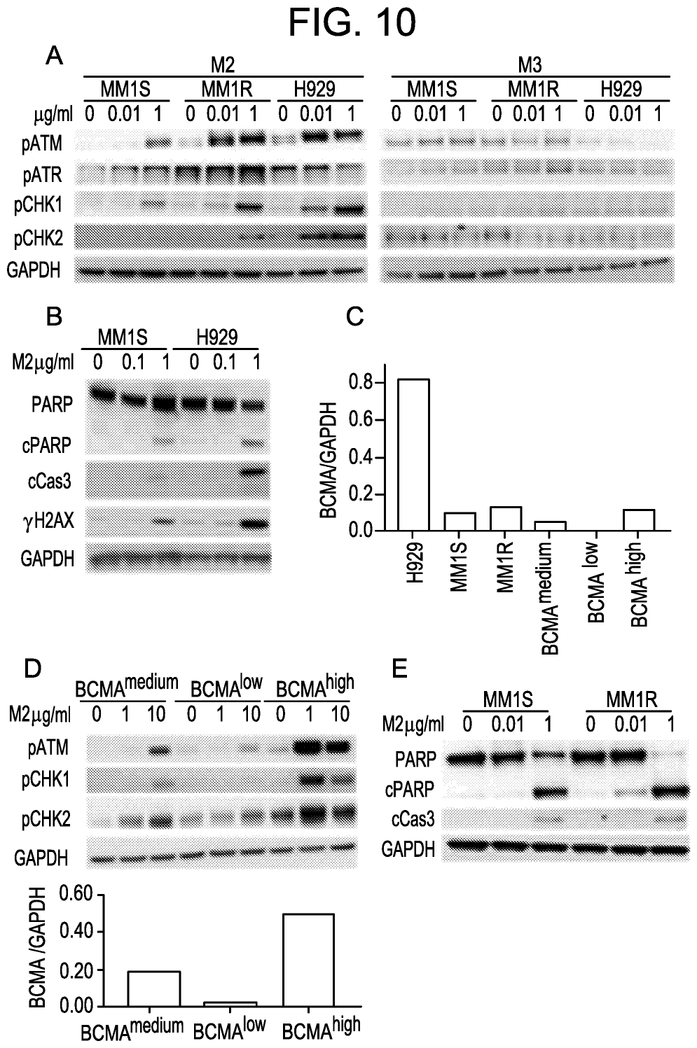

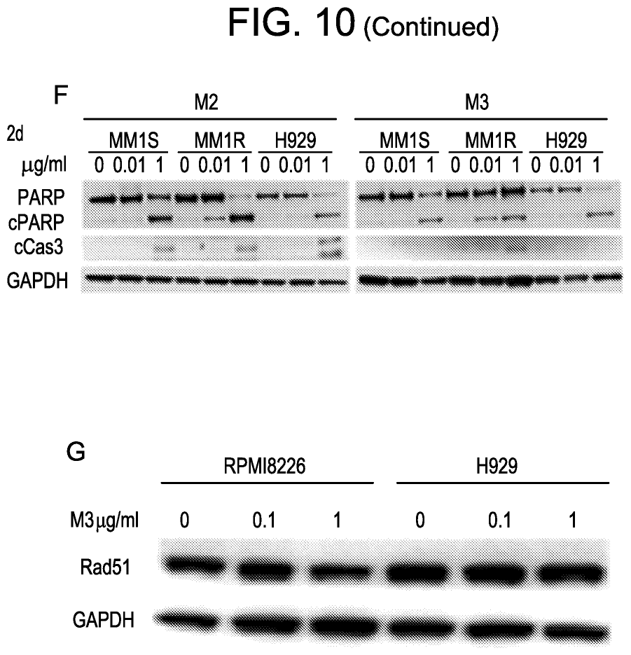

Immunoblot analysis was used to determine induction of DNA damage response (DDR) signalling cascades triggered by M2 in MM cell lines in a time- and dose-dependent manner. M2, but not M3, incudes phosphorylation of ATM, cell cycle checkpoint kinase 1 (CHK1), and CHK2 (CHK1/2), as well as histone 2AX (H2AX), an early event in the DNA double strand break (DSB) response (FIGS. 9A and 10A). M2-stimulated phosphorylation of ATM and CHK1/2 is detected at 4 h and sustained for >1 d following treatment. Earlier and more pronounced activation of ATM and CHK1/2 triggered by M2 was seen in H929 cells which express significantly higher BCMA levels than MM1 S cells (FIG. 10A, C). The intensity of M2-induced phosphorylation of ATM and CHK1/2 also correlated with BCMA levels derived from the parental RPM18226 MM cells (FIG. 10D). M2-induced phosphorylation of ATM and CHK1/2 occurs to a significantly greater extent than phosphorylation of ATR, in MM cells tested. Following 2d-treatment with M2, cleavage of PARP (cPARP) and caspase 3 (cCas3) is induced in a BCMA-dependent manner (FIG. 10B, E-F), associated with increased phosphorylated H2AX (yH2AX) (FIG. 10B), indicating that M2 induces DNA damages followed by apoptosis in MM cells. Importantly, M2 dose-dependently induces phosphorylation of ATM and CHK1/2 in all MM cells, including 6 cell lines with p53 mutations (FIG. 9b-c). Under the same treatment conditions as M2, M3 induce neither ATM/ATR nor CHK1/2 (FIG. 10A). M2 is more effective than M3 in inducing cPARP and cCas3 (FIG. 10F), consistent with its higher potency than M3 in triggering MM cell apoptosis. Significantly, M2 triggered ATM/ATR and downstream CHK1/2 signaling pathways, yH2AX and PARP cleavage in ANBL6 and the paired btz-resistant ANBL6-BR cells (FIG. 9c). Prominent activation of ATM and CHK1/2 by M2 is also seen in IMiD-resistant H929(R) cells to a similar extent as the parental H929 MM cells (FIG. 9d). Thus, in btz- and len-resistant MM cells, M2 still induces BCMA-dependent DDR signaling pathways via activation of ATR/ATM-CHK1/2 signaling cascade, followed by apoptosis.

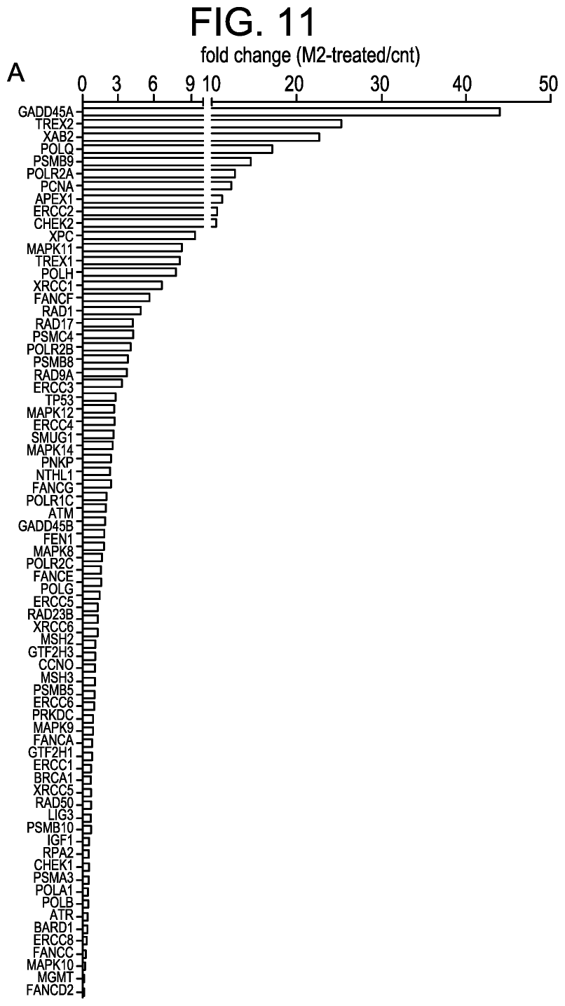

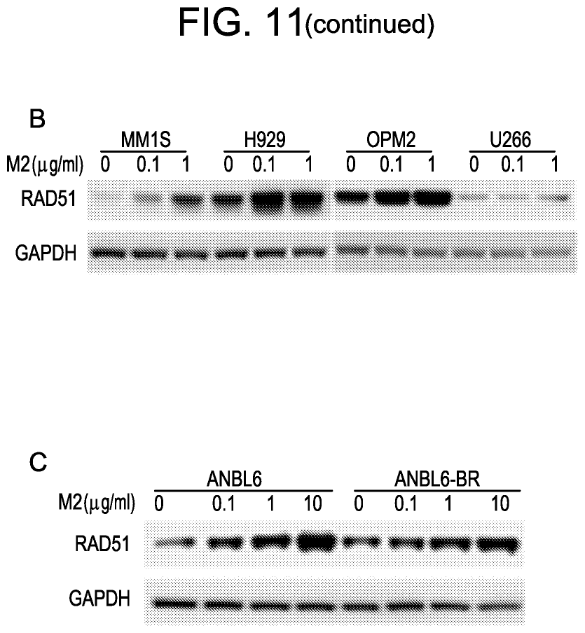

Analysis of DNA repair mechanism TagMan® array show that M2 changes expression of DNA damage repair-associated genes (51 out of 72) in H929 MM cells (FIG. 11a). In various drug-sensitive and -resistant MM cells (n>6), M2, in a dose-dependent fashion, induces RAD51, which binds to DNA ICLs before a DSB is generated (FIG. 11 b-c), whereas M3 does not (FIG. 10G). Thus, in MM cells, M2 specifically activates ATM/ATR-CHK1/2-mediated DDR signalling cascades and induces downstream DDR-related molecules associates with increased yH2AX and RAD51, followed by apoptosis.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

SEQUENCES

| | | SEQ |
|---|---|---|
| Identifier | Amino Acid Sequence | ID NO: |
| Heavy Chain | SYSMN | 1 |
| CDR1 (HCDR1) | | |
| HCDR2 | SISGSSNYIYYADSVKG | 2 |
| HCDR3 | GGNYYVEYFQY | 3 |
| Light Chain | RASQYISSNYLA | 4 |
| CDR1 (LCDR1) | | |
| LCDR2 | GASNRAT | 5 |
| LCDR3 | QQYGSSPIT | 6 |
| VH (germlined) | EVQLVESGGGLVKPGGSLRLSC | 7 |
| | AASGFTFRSYSMNWVRQAPGKG | |
| | LEWVSSISGSSNYIYYADSVKG | |
| | RFTISRDNAKNSLYLQMNSLRA | |
| | EDTAVYYCARGGNYYVEYFQYW | |
| | GQGTLVTVSS | |
| VL (germlined) | EIVLTQSPGTLSLSPGERATLS | 8 |
| | CRASQYISSNYLAWYQQKPGQA | |
| | PRLLIYGASNRATGIPDRFSGS | |
| | GSGTDFTLTISRLEPEDFAVYY | |
| | CQQYGSSPITFGQGTKLEIK | |
| VH (WT) | EIQLVESGGGLVKPGGSLRLSC | 9 |
| | AASGFTFRSYSMNWVRQAPGKG | |
| | LEWVSSISGSSNYIYYADSVKG | |
| | RFTISRDNAKNSLYLQMNSLRA | |
| | EDTALYYCARGGNYYVEYFQYW | |
| | GQGTLVTVSS | |
| VL (WT) | EIVLTQSPGTLSLSPGERATLS | 10 |
| | CRASQYISSNYLAWYQQKPGQA | |
| | PRLLIYGASNRATGIPDRFSGS | |
| | GSGTGFTLTISRLEPEDFAVFY | |
| | CQQYGSSPITFGQGTKLEIK | |
| Heavy Chain | ASTKGPSVFPLAPSSKSTSGGT | 11 |
| constant region: | AALGCLVKDYFPEPVTVSWNSG | |
| (Cysteine | ALTSGVHTFPAVLQSSGLYSLS | |
| insertion | SVVTVPSSSLGTQTYICNVNHK | |
| underlined): | PSNTKVDKRVEPKSCDKTHTCP | |
| | PCPAPELLGGPSCVFLFPPKPK | |
| | DTLMISRTPEVTCVVVDVSHED | |
| | PEVKFNWYVDGVEVHNAKTKPR | |
| | EEQYNSTYRVVSVLTVLHQDWL | |
| | NGKEYKCKVSNKALPAPIEKTI | |
| | SKAKGQPREPQVYTLPPSREEM | |
| | TKNQVSLTCLVKGFYPSDIAVE | |
| | WESNGQPENNYKTTPPVLDSDG | |
| | SFFLYSKLTVDKSRWQQGNVFS | |
| | CSVMHEALHNHYTQKSLSLSPG | |
| | K | |
| Human Kappa | RTVAAPSVFIFPPSDEQLKSGT | 12 |
| Light Chain | ASVVCLLNNFYPREAKVQWKVD | |
| | NALQSGNSQESVTEQDSKDSTY | |
| | SLSSTLTLSKADYEKHKVYACE | |
| | VTHQGLSSPVTKSFNRGEC | |
| Human BCMA | MLQMAGQCSQNEYFDSLLHACI | 13 |
| sequence; | PCQLRCSSNTPPLTCQRYCNAS | |
| UniProtKB- | VTNSVKGTNAILWTCLGLSLII | |
| Q02223 | SLAVFVLMFLLRKINSEPLKDE | |
| | FKNTGSGLLGMANIDLEKSRTG | |
| | DEIILPRGLEYTVEECTCEDCI | |
| | KSKPKVDSDHCFPLPAMEEGAT | |
| | ILVTTKTNDYCKSLPAALSATE | |
| | IEKSISAR | |

1. A B-cell malignancy medicament, comprising:
a. an antibody-drug conjugate (ADC) comprising an antibody or antigen-binding fragment thereof that binds to B-cell maturation antigen (BCMA), conjugated to a nucleic acid cross-linking agent; and
b. a proteasome inhibitor;
wherein the medicament provides an enhanced suppression of a B-cell malignancy when compared with an otherwise identical medicament lacking said proteasome inhibitor; or
wherein the medicament provides an enhanced suppression of a B-cell malignancy when compared with an otherwise identical medicament lacking said ADC.

2. A therapeutic combination for use in treating a B-cell malignancy, said therapeutic combination comprising:
a. an ADC comprising an antibody or antigen-binding fragment thereof that binds to BCMA, conjugated to a nucleic acid cross-linking agent; and
b. a proteasome inhibitor;
wherein the therapeutic combination provides an enhanced suppression of a B-cell malignancy when compared with an otherwise identical composition lacking said proteasome inhibitor; or
wherein the therapeutic combination provides an enhanced suppression of a B-cell malignancy when compared with an otherwise identical composition lacking said ADC.

3. A method for treating a B-cell malignancy, the method comprising administering to a subject a therapeutic combination comprising:
a. an ADC comprising an antibody or antigen-binding fragment thereof that binds to BCMA, conjugated to a nucleic acid cross-linking agent; and
b. a proteasome inhibitor;
wherein the therapeutic combination provides an enhanced suppression of a B-cell malignancy when compared with an otherwise identical composition lacking said proteasome inhibitor; or
wherein the therapeutic combination provides an enhanced suppression of a B-cell malignancy when compared with an otherwise identical composition lacking said ADC.

4. An in vitro method for enhancing ADC suppression of a malignant B-cell, said method comprising contacting a malignant B-cell with (a) an ADC comprising an antibody or antigen-binding fragment thereof that binds to BCMA, conjugated to a nucleic acid cross-linking agent, in combination with (b) a proteasome inhibitor.

5. An in vitro method for enhancing proteasome inhibitor suppression of a malignant B-cell, said method comprising contacting a malignant B-cell with (a) a proteasome inhibitor, in combination with (b) an ADC comprising an antibody or antigen-binding fragment thereof that binds to BCMA, conjugated to a nucleic acid cross-linking agent.

6. The medicament, therapeutic combination for use, method or in vitro method according to any one of the preceding claims:
a. wherein said proteasome inhibitor is administered prior to, simultaneously with or sequentially to said ADC; or
b. wherein said ADC is administered prior to, simultaneously with or sequentially to said proteasome inhibitor.

7. The medicament, therapeutic combination for use, method or in vitro method according to any one of the preceding claims, wherein said B-cell malignancy is characterised by comprising a malignant B-cell having an increased expression level of BCMA antigen relative to a reference non-malignant B-cell.

8. The medicament, therapeutic combination for use, method or use according to any one of the preceding claims, wherein said B-cell malignancy is one or more selected from B-cell lymphoma, B-cell leukaemia, myeloma, multiple myeloma or a combination thereof.

9. The medicament, therapeutic combination for use, method or use according to any one of the preceding claims, wherein said B-cell malignancy is multiple myeloma.

10. The medicament, therapeutic combination for use, method or use according to any one of the preceding claims, wherein said antibody or antigen binding fragment thereof comprises the following six CDRs:
a. a heavy chain CDR1 comprising an amino acid sequence of SEQ ID NO: 1, or a functional variant thereof;
b. a heavy chain CDR2 comprising an amino acid sequence of SEQ ID NO: 2, or a functional variant thereof;
c. a heavy chain CDR3 comprising an amino acid sequence of SEQ ID NO: 3, or a functional variant thereof;
d. a light chain CDR1 comprising an amino acid sequence of SEQ ID NO: 4, or a functional variant thereof;
e. a light chain CDR2 comprising an amino acid sequence of SEQ ID NO: 5, or a functional variant thereof; and
f. a light chain CDR3 comprising an amino acid sequence of SEQ ID NO: 6, or a functional variant thereof.

11. The medicament, therapeutic combination for use, method or use according to any one of the preceding claims, wherein said antibody or antigen binding fragment thereof comprises:
a. a heavy chain variable region comprising an amino acid sequence of SEQ ID: NO 7, or a functional equivalent thereof; and/or
b. a light chain variable region comprising an amino acid sequence of SEQ ID: NO 8, or a functional equivalent thereof.

12. The medicament, therapeutic combination for use, method or use according to any one of the preceding claims, wherein the antibody or antigen binding fragment thereof comprises a heavy chain constant region comprising a cysteine (C) insertion between the serine (S) at position 239 and the valine (V) at position 240, wherein the numbering corresponds to the EU index in Kabat.

13. The medicament, therapeutic combination for use, method or use according to any one of the preceding claims, wherein the antibody or antigen binding fragment thereof comprises a heavy chain constant region comprising the amino acid sequence of SEQ ID NO: 11.

14. The medicament, therapeutic combination for use, method or use according to any one of the preceding claims, wherein the antibody or antigen binding fragment thereof comprises a human kappa constant region comprising the amino acid sequence of SEQ ID NO: 12.

15. The medicament, therapeutic combination for use, method or use according to any one of the preceding claims, wherein the proteasome inhibitor is one or more selected from bortezomib, carfilzomib, ixazomib, marizomib, oprozomib, delanzomib, or a combination thereof.

16. The medicament, therapeutic combination for use, method or use according to any one of the preceding claims, wherein the proteasome inhibitor is bortezomib.

17. The medicament, therapeutic combination for use, method or use according to any one of the preceding claims, wherein said nucleic acid cross-linking agent is a pyrrolobenzodiazepine (PBD).

18. The medicament, therapeutic combination for use, method or use according to any one of the preceding claims, wherein said nucleic acid cross-linking agent is a PBD of one or more selected from (a) SG3249, (b) SG3315 or (c) SG3400, comprising the formula: 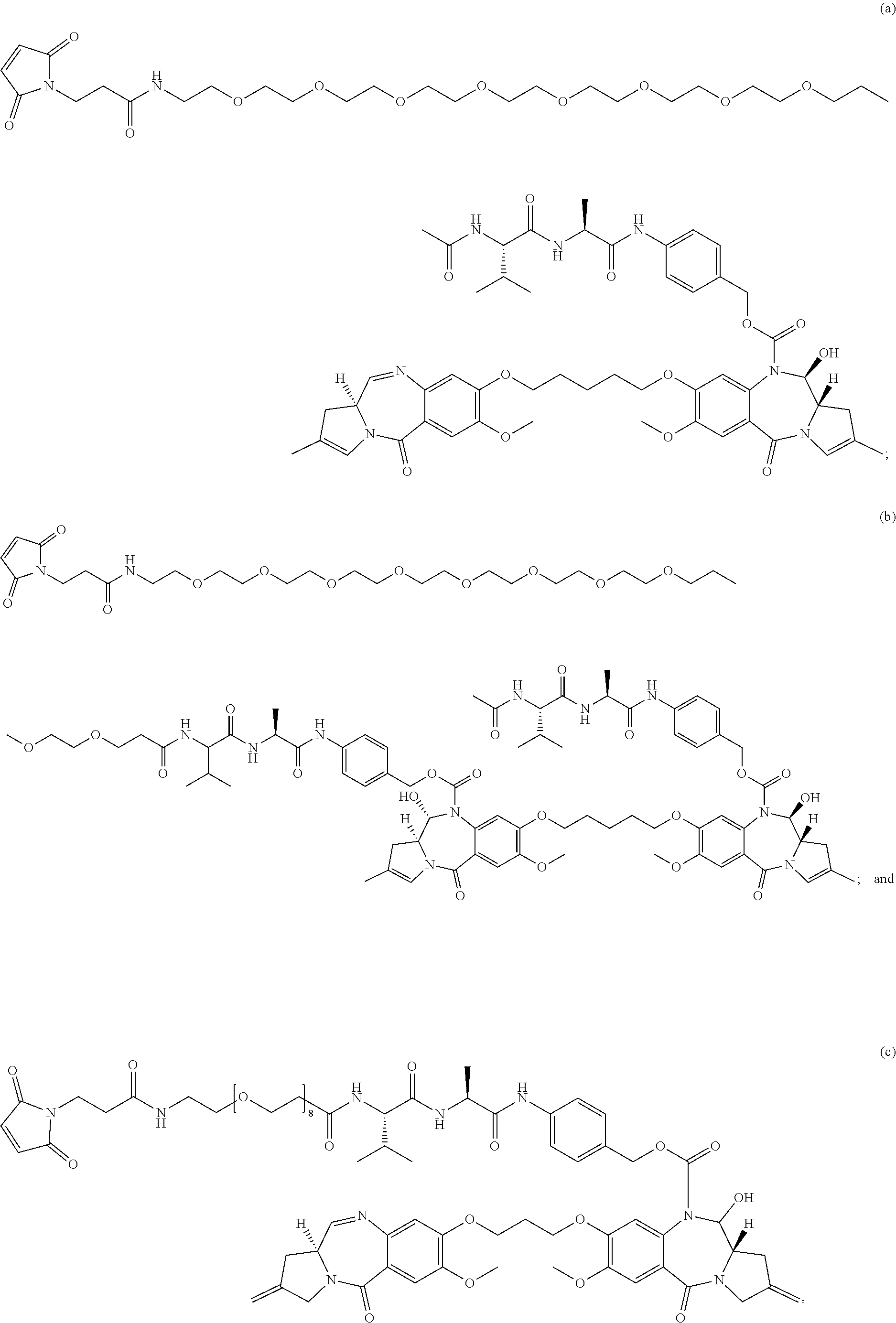
respectively, or a combination thereof.

19. The medicament, therapeutic combination for use, method or use according to any one of the preceding claims, wherein said nucleic acid cross-linking agent is the PBD SG3249, comprising the formula:

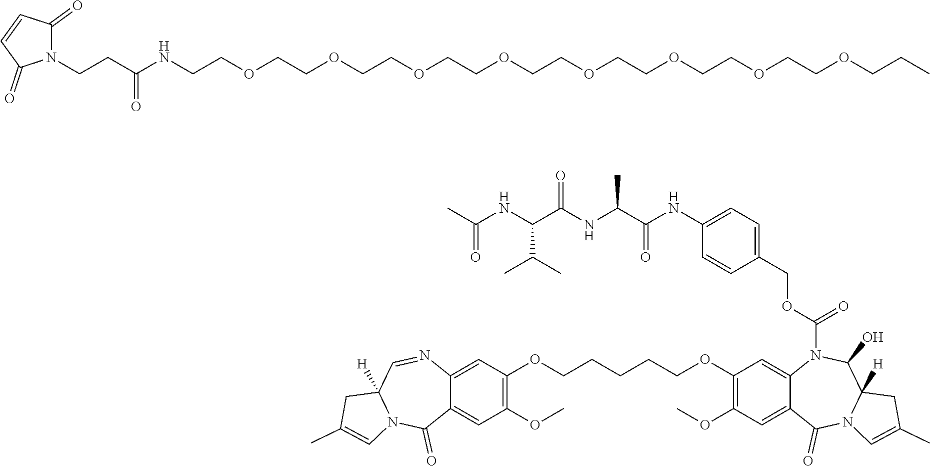

20. The medicament, therapeutic combination for use, method or use according to any one of the preceding claims, wherein said antibody or antigen binding fragment thereof is a monoclonal antibody.

21. The medicament, therapeutic combination for use, method or use according to any one of the preceding claims, wherein the medicament or therapeutic combination comprises a pharmaceutically acceptable carrier.

22. The medicament, therapeutic combination for use, method or use according to any one of the preceding claims, wherein the B-cell malignancy is resistant to one or more selected from dexamethasone, lenalidomide, pomalidomide, bortezomib, or a combination thereof.

23. The medicament, therapeutic combination for use, method or use according to any one of the preceding claims, wherein the B-cell malignancy is resistant to bortezomib.

24. The medicament, therapeutic combination for use, method or use according to any one of the preceding claims, wherein said enhanced suppression of a B-cell malignancy comprises one or more selected from an enhanced delay in tumour growth, an enhanced reduction in tumour size, an enhanced reduction in tumour metastasis, an enhanced survival rate in a subject comprising a B-cell malignancy, or a combination thereof.

25. The medicament, therapeutic combination for use, method or use according to any one of the preceding claims, wherein the medicament or therapeutic combination is administered by intravenous infusion.


SEQUENCE LISTING (13 sequences)

SEQ ID NO: 1 - protein, 5 aa, Artificial Sequence
  Synthetic
SYSMN

SEQ ID NO: 2 - protein, 17 aa, Artificial Sequence
  Synthetic
SISGSSNYIYYADSVKG

SEQ ID NO: 3 - protein, 11 aa, Artificial Sequence
  Synthetic
GGNYYVEYFQY

SEQ ID NO: 4 - protein, 12 aa, Artificial Sequence
  Synthetic
RASQYISSNYLA

SEQ ID NO: 5 - protein, 7 aa, Artificial Sequence
  Synthetic
GASNRAT

SEQ ID NO: 6 - protein, 9 aa, Artificial Sequence
  Synthetic
QQYGSSPIT

SEQ ID NO: 7 - protein, 120 aa, Artificial Sequence
  Synthetic
EVQLVESGGGLVKPGGSLRLSCAASGFTFRSYSMNWVRQAPGKGLEWVSSISGSSNYIYYADSVKGRFTISRDNAKNSLYLQMNSLRAEDTAVYYCARGGNYYVEYFQYWGQGTLVTVSS

SEQ ID NO: 8 - protein, 108 aa, Artificial Sequence
  Synthetic
EIVLTQSPGTLSLSPGERATLSCRASQYISSNYLAWYQQKPGQAPRLLIYGASNRATGIPDRFSGSGSGTDFTLTISRLEPEDFAVYYCQQYGSSPITFGQGTKLEIK

SEQ ID NO: 9 - protein, 120 aa, Artificial Sequence
  Synthetic
EIQLVESGGGLVKPGGSLRLSCAASGFTFRSYSMNWVRQAPGKGLEWVSSISGSSNYIYYADSVKGRFTISRDNAKNSLYLQMNSLRAEDTALYYCARGGNYYVEYFQYWGQGTLVTVSS

SEQ ID NO: 10 - protein, 108 aa, Artificial Sequence
  Synthetic
EIVLTQSPGTLSLSPGERATLSCRASQYISSNYLAWYQQKPGQAPRLLIYGASNRATGIPDRFSGSGSGTGFTLTISRLEPEDFAVFYCQQYGSSPITFGQGTKLEIK

SEQ ID NO: 11 - protein, 331 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 107 aa, Artificial Sequence [sequence omitted]

SEQ ID NO: 13 - protein, 184 aa, Artificial Sequence
  Synthetic
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR